(19) United States
(12) Patent Application Publication
(10) Pub. No.: US 2004/0096826 A1
(43) Pub. Date: May 20, 2004

(54) Methods for creating recombination products between nucleotide sequences

(76) Inventor: Glen A. Evans, San Marcos, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/062,188

(22) Filed: Jan. 30, 2002

(51) Int. Cl. C12Q 1/68; H01L 21/00

(52) U.S. Cl. 435/6; 438/1

(57) ABSTRACT

The invention is directed to the creation of a collection of recombination products between two or more nucleotide sequences. The nucleotide sequences can encode distinct amino acid sequences and the collection of recombination products can be expressed to obtain a corresponding collection of polypeptide recombination products or variants. The amino acid sequences encoded by the two or more nucleotide sequences can correspond to polypeptides that are similar in function, but are encoded by dissimilar nucleotide sequences that cannot be recombined using traditional methods of recombination, which require a high degree of sequence similarity.

### BACKGROUND OF THE INVENTION

The present invention relates to the field of synthetic gene technology and, more specifically, to a method for generating a collection of recombination products between distinct nucleotide sequences.

A protein having a specific bioactivity exhibits sequence variation not only between genera, but often differences even exist between members of the same species. This variation is most pronounced at the genomic level and the natural genetic diversity among genes coding for proteins having basically the same bioactivity has been generated in nature over billions of years and can reflect a natural optimization of the proteins coded for in respect of the environment of the particular host organism. Nevertheless, naturally occurring bioactive molecules often are not optimized for the various uses to which they are put by mankind, such that a need exists to identify bioactive proteins that exhibit optimal properties in respect to its intended use.

For many years, optimization of bioactivity has been attempted by screening of natural sources, or by use of mutagenesis. In particular, site-directed mutagenesis results in substitution, deletion or insertion of specific amino acid residues chosen either on the basis of their type or on the basis of their location in the secondary or tertiary structure of the mature enzyme.

One method for the recombination between two or more nucleotide sequences of interest involves shuffling homologous DNA sequences by using in vitro Polymerase Chain Reaction (PCR) methods. Nucleic acid recombination products containing shuffled nucleotide sequences are selected from a DNA library based on the improved function of the expressed proteins. A disadvantage inherent to this method is its dependence on the use of homologous gene sequences and the production of random fragments by cleavage of the template double-stranded polynucleotide. In particular, because recombination has to be performed among nucleotide sequences with sufficient sequence homology to enable hybridization of the different sequences to be recombined, the inherent disadvantage is that the diversity generated is relatively limited. Other methods rely on the presence of conserved sequence regions and, therefore, also require a sufficient degree of homology between the sequences to be recombined. While methods exist for making recombinant cloned libraries containing shuffled proteins of similar sequence, there is no current way of creating a collection of recombination products where the sequence is less than forty percent identical.

Thus, there exists a need for a method of making recombination products of proteins that are similar in tertiary structure, but encoded by dissimilar nucleotide sequences. The present invention satisfies this need and provides related advantages as well.

### SUMMARY OF THE INVENTION

The invention is directed to a method of creating a collection of recombination products between two nucleotide sequences by combining an initial set of oligonucleotides corresponding to a first nucleotide sequence with a subsequent set of oligonucleotides corresponding to a distinct nucleotide sequence and one or more sets of combination oligonucleotides containing a nucleotide sequence region corresponding to the initial nucleotide sequence region and further containing a nucleotide sequence region corresponding to the subsequent nucleotide sequence.

In one embodiment, the invention provides a method of creating a collection of recombination products between two or more nucleotide sequences that includes the steps of (a) generating an initial set of oligonucleotides corresponding to a first nucleotide sequence and one or more subsequent sets of oligonucleotides, each corresponding to a distinct nucleotide sequence; (b) generating one or more sets of combination oligonucleotides, each containing a nucleotide sequence corresponding to the initial nucleotide sequence and further including a nucleotide sequence corresponding to at least one of the subsequent nucleotide sequences; and (c) assembling a collection of polynucleotide recombination products by combining the oligonucleotides corresponding to each of the sets. If desired, the initial and the subsequent nucleotide sequences can each encode a distinct amino acid sequence and the collection of recombination products can be expressed to obtain a corresponding collection of polypeptide variants. In addition, the recombination products can be single or multiple recombination products.

### BRIEF DESCRIPTION OF THE DRAWINGS

FIG. 1 the amino acid sequences of (A) E. Cloacae [SEQ ID NO:1] (B) K. pneumoniae [SEQ ID NO:2], and (C) an example of a polypeptide variant [SEQ ID NO:3] encoded by a polynucleotide recombination product between the corresponding E. Cloacae and K. pneumoniae nucleotide sequences.

FIG. 2 shows a schematic of the assembly scheme for single recombination products between E. Cloacae and K. pneumoniae nucleotide sequences.

FIG. 3 shows a schematic of the assembly scheme for all possible recombination products between E. Cloacae and K. pneumoniae nucleotide sequences.

FIG. 4 shows(A)the nucleotide sequence [SEQ ID NO:4] and corresponding amino acid sequence [SEQ ID NO:5] of AF169027, (B) the nucleotide sequence [SEQ ID NO:6] and corresponding amino acid sequence [SEQ ID NO:7] of HSA225092, (C) the AF169027 and HSA225092 amino acid sequences shortened by truncation [SEQ ID NOS:8 and 9, respectively] to make two sequences of equal length, and (D) synthetic AF169027 and HSA225092 genes [SEQ ID NOS:10 and 42, respectively] derived based on E.coli codon preferences.

FIG. 5 shows (A) the amino acid sequence of a butterfly biliverdin binding protein BBP-B1X [SEQ ID NO:104], and (B) the amino acid sequence of the human Retinoic Acid binding protein (RA BP) [SEQ ID NO:105].

FIG. 6 shows a schematic representation of AF169027 is a single chain mouse monoclonal antibody that combines a V_{H} and V_{L} chain with a peptide linker.

FIG. 7 shows a schematic of the assembly scheme for all possible recombination products between the AF169027 and HSA225092 nucleotide sequences.

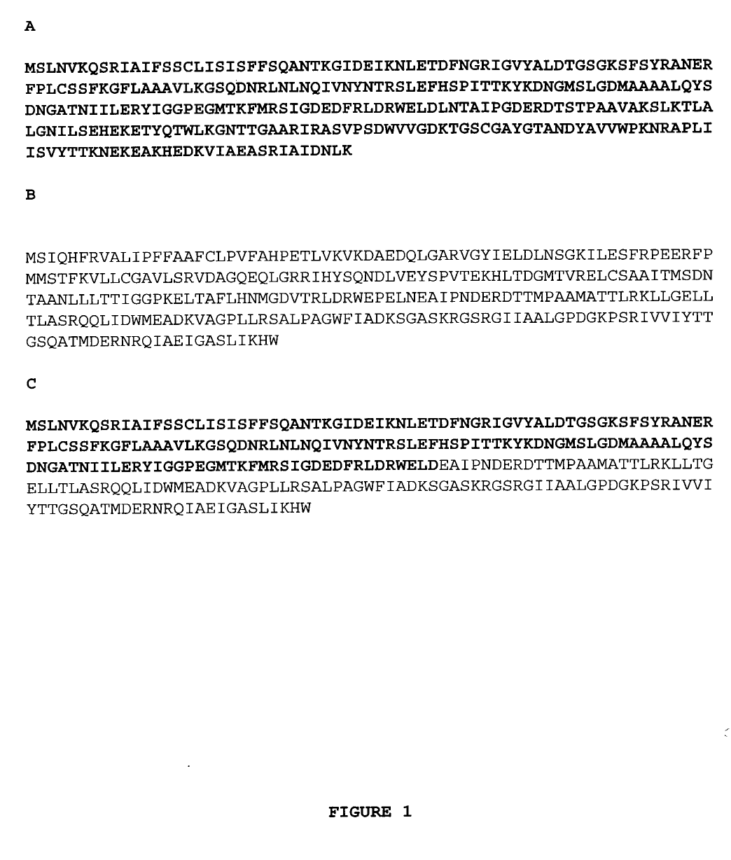

### DETAILED DESCRIPTION OF THE INVENTION

The invention is directed to the creation of a collection of recombination products between two or more nucleotide sequences. The nucleotide sequences can encode distinct amino acid sequences and the collection of polynucleotide recombination products can be expressed to obtain a corresponding collection of polypeptide recombination products or variants. The amino acid sequences encoded by the two or more nucleotide sequences can correspond to polypeptides that have similar function, but are encoded by dissimilar nucleotide sequences which cannot be recombined using traditional methods of recombination that require a high degree of sequence similarity.

The invention method for assembling a collection library or population of polypeptide variants that correspond to single or multiple recombination products between two or more nucleotide sequences is predicated on the idea that by being able to achieve recombination independent of sequence similarity between the sequences to be recombined, it is possible for the user to design a desired recombination product without being limited by a requirement for sequence similarity. The invention method thus provides the ability to design and synthesize a collection of recombination products between two or more distinct nucleotide sequences based on any criteria desired by the user.

In one embodiment, the invention is directed to a method of creating a collection of single or multiple recombination products between genes that encode polypeptides of similar tertiary structure, but dissimilar sequence.

In another embodiment, the invention is directed to a method of creating a collection of single or multiple recombination products between genes that encode polypeptides of similar tertiary structure and similar sequence.

Id a particular embodiment, the methods of the invention can be used to create a collection of polynucleotide recombination products that correspond to distinct antibody molecules each having, for example, a distinct complementarity determining region (CDR). In this embodiment, the invention method enables the user to produce a collection of recombination products corresponding to synthetic antibodies or antibody like molecules through the directed recombination methods described herein.

As used herein, the term “polynucleotide recombination product” refers to a polynucleotide that, as a result of synthetic recombination via the invention method, contains sequence regions corresponding to two or more distinct nucleotide sequences. In the methods of the invention, polynucleotide recombination products are assembled from initial and subsequent sets of oligonucleotides and one or more sets of combination oligonucleotides. Polynucleotide recombination products can be single, double or multiple recombination products, depending on the oligonucleotide sets from which they are assembled as well as on the algorithm of assembly.

A “single recombination product,” as defined herein, has one juncture, which also can be referred to as a breakpoint or border, between distinct nucleotide sequences that are recombined, such that the product has a 3′ region, also referred to as a 3′ portion, corresponding to a first nucleotide sequence and a 5′ region, also referred to as a 5′ portion, corresponding to a subsequent nucleotide sequence. A “multiple recombination product” has two or more junctures, which also can be referred to as breakpoints or borders, between distinct nucleotide sequences that are recombined. For example, a double recombination product can have two junctures such that the 3′ and 5′ regions or portions correspond to the same nucleotide sequence, which flanks a distinct sequence.

As used herein, the term “oligonucleotide” refers to a molecule that encompasses two or more deoxyribonucleotides or ribonucleotides. Oligonucleotides are nucleotide segments, single-stranded or double-stranded, consisting of the nucleotide bases linked via phosphodiester bonds. Nucleotides are present in either DNA or RNA and encompass adenosine (A), guanine (G), cytosine (C) or thymine (T) or uracil (U), respectively, as base, and a sugar moiety being deoxyribose or ribose, respectively. An oligonucleotide also can contain modified bases or bases other than adenosine (A), guanine (G), cytosine (C) or thymine (T) or uracil (U) such as, for example, 8-azaguanine and hypoxanthine. Modifications include, for example, derivatization and covalent attachment with chemical groups. Other bases can include, for example, pyrimidine or purine analogs, precursors such as inosine that are capable of base pair formation, and tautomers. Similarly, an oligonucleotide also can contain modified or derivative forms of the ribose or deoxyribose sugar moieties, including, for example, functional analogs thereof. Those skilled in the art will know what natural or non-naturally occurring nucleotide, nucleoside or base forms can be incorporated into an oligonucleotide, including derivatives and analogs. If desired the nucleotides can carry a label or marker to allow detection. Exemplary labels include a radioisotope, a fluorophore, a calorimetric agent, a magnetic substance, an electron-rich material such as a metal, a luminescent tag, an electrochemiluminescent label, or a binding agent such as biotin. Specific examples of labels for use in detecting nucleotides are known in the art as are methods for incorporating labels.

A plus strand or 5′ oligonucleotide, by convention, includes a single-stranded polynucleotide segment that starts with the 5′ end to the left as one reads the sequence. A minus strand or 3′ oligonucleotide includes a single-stranded polynucleotide segment that starts with the 3′ end to the left as one reads the sequence. A set of oligonucleotides useful in the methods of the invention can encompass oligonucleotides corresponding to either or both a plus and a minus strand.

As used herein, the term “combination oligonucleotide” refers to an oligonucleotide that contains sequence regions from two or more distinct nucleic acid molecules that are subject to recombination via the invention method. A combination oligonucleotide will encompass a sequence region of at least between about 5 and 25 nucleotides, between about 6 and 15 nucleotides, between about 7 and 12 nucleotides, between about 8 and 10 nucleotides corresponding to each of the first and subsequent nucleotide sequences that are recombinant via the invention method. A combination oligonucleotide can, for example, encompass a 3′ region corresponding to one nucleotide sequence and a 5′ region corresponding to a distinct nucleotide sequence. A set of combination oligonucleotides further can represent a plus or minus strand, also referred to as a forward and a reverse strand combined from two distinct double-stranded nucleotide sequences where each oligonucleotide contains a sequence region corresponding to each of the nucleotide sequences. Thus, a sequence region contained in a combination oligonucleotide can correspond to a first or a subsequent nucleotide sequence of the invention and can encompass at least 6, at least 7, at least 8, at least 9, at least 10, at least 11, at least 12, at least 13, at least 14, at least 15, at least 17, at least 18, at least 19, at least 20, at least 21, at least 22, at least 23, at least 24, at least 25 or more nucleotides corresponding to the reference nucleotide sequence.

As used herein, the term “assembling” refers to the process of constructing a polynucleotide recombination product using as components the oligonucleotides of the initial and subsequent sets and the one or more set of combination oligonucleotides. To assemble a polynucleotide recombination product, oligonucleotides of the initial and subsequent sets can be mixed with the one or more sets of combination oligonucleotides according to a variety of mixing schemes, for example, triplex mixing.

As described herein, the initial and subsequent sets and the set of combination oligonucleotides can be parsed by computer, the information can be used to direct the synthesis of arrays of oligonucleotides, for example, in microtiter plates and the sets of arrayed sequences subsequently can be assembled using a mixed pooling strategy that includes a desired mixing scheme or algorithm, for example, triplet mixing or any desired mixing schemes involving mixing of more than three oligonucleotides to prepare intermediates corresponding to, for example, five-plexes, seven-plexes, nine-plexes or eleven-plexes of oligonucleotides.

Homologous recombination plays two important roles in the life cycle of most organisms. Recombination generates diversity by creating new combinations of genes, or parts of genes. It is also required for genome stability as it is essential for the repair of some types of DNA lesions in mitotic cells and for segregation of homologous chromosomes during meiosis. The importance of the latter functions is evidenced by increased mutagenesis, and mitotic and meiotic aneuploidy in the absence of recombination functions.

Naturally occurring homologous recombination is a cellular process that results in the scission of two nucleotide sequences having identical or substantially similar or “homologous” sequences and the ligation of the two sequences following crossover. The result is that one region of each initially present sequence becomes ligated to a region of the other initially present sequence as described by Sedivy, Bio-Technology 6:1192-1196 (1988), which is incorporated herein by reference. Homologous recombination is, thus, a sequence specific process by which cells can transfer a portion of sequence from one DNA molecule to another. The portion can be of any length from several bases to a substantial fragment of a chromosome.

For homologous recombination to naturally occur between two nucleotide sequences, the molecules need to possess a region of sequence similarity with respect to one another. Naturally occurring homologous recombination is catalyzed by enzymes which are naturally present in both prokaryotic and eukaryotic cells. The transfer of a region of nucleotide sequence can be envisioned as occurring through a multi-step process. If a particular region is flanked by regions of homology, then two recombinational events can occur and result in the exchange of a region between two nucleotide sequences. Recombination can be reciprocal, and thus result in an exchange of regions between two recombining nucleotide sequences. The frequency of natural recombination between two nucleotide sequences can be enhanced by treatment with agents which stimulate recombination such as trimethylpsoralen or UV light.

Recombination between homologous genes is one method for generating sequence diversity, and can be applied to protein analysis and directed evolution. In vitro recombination methods such as DNA shuffling can produce hybrid genes with multiple crossovers and has been used to evolve proteins with improved and new properties. Recently in vivo recombination has been used to generate diversity for directed evolution, for example, creation of large phage display antibody libraries. The methods for preparing a collection of recombination products provided by the invention, which allow for recombination independent of sequence similarity and based on any criteria desired by the user, can be applied to exploit the recently gained abundance in genomic sequence data and enhances the potential for preparing engineered polypeptide variants.

The present invention is directed to the discovery that recombination products between nucleotide sequences that encode polypeptides of similar tertiary structure, but having dissimilar sequence can be created using gene synthesis methods as described herein. By designing and assembling a collection of polynucleotide recombination products via the methods of the invention it is possible to create recombination products between polypeptides having a sequence identity of less than 95%, less than 90%, less than 80%, less than 70%, less than 60%, less than 50%, less than 40%, less than 30% or less than 20%.

The invention provides a method of creating a collection of recombination products between two or more nucleotide sequences by combining an initial set of oligonucleotides corresponding to a first nucleotide sequence with a subsequent set of oligonucleotides corresponding to a distinct subsequent nucleotide sequence and one or more sets of combination oligonucleotides encompassing a nucleotide sequence region corresponding to the initial nucleotide sequence and further encompassing a nucleotide sequence region corresponding to the subsequent nucleotide sequence.

In one embodiment, the invention provides a method of creating a collection of recombination products between two or more nucleotide sequences including the steps of (a) generating an initial set of oligonucleotides corresponding to a first nucleotide sequence and one or more subsequent sets of oligonucleotides, each of the subsequent sets corresponding to a distinct subsequent nucleotide sequence; (b) generating one or more sets of combination oligonucleotides, each of the combination oligonucleotides encompassing a sequence region corresponding to the initial nucleotide sequence and further encompassing a sequence region corresponding to at least one of the one or more subsequent nucleotide sequences; and (c) assembling a collection of polynucleotide recombination products by combining oligonucleotides corresponding to each of the sets. The initial and subsequent sets of oligonucleotides can correspond to nucleic sequences that encode distinct amino acid sequences.

The collection of polynucleotide recombination products prepared by the invention method can further be expressed to prepare a corresponding collection or library of polypeptide variants. Furthermore, the invention can be practiced by performing the initial step of selecting amino acid sequences and subsequently preparing sets of oligonucleotides that correspond to nucleotide sequences which encode the selected amino acid sequences as is shown in the Examples that follow. However, while the polynucleotide recombination products can be selected or targeted based on the corresponding variant polypeptides they encode, the methods of the invention can be practiced with nucleotide sequences regardless of whether they are encoding or non-encoding.

Thus, the invention also provides a method for assembling a library, or a population or a collection of polypeptide variants that correspond to single or multiple polynucleotide recombination products between two or more nucleotide sequences. The invention method allows for recombination independent of sequence similarity between the sequences to be recombined and enables the user to design a desired recombination product without being limited by a requirement for sequence similarity. The invention method thus provides the ability to design and synthesize a collection of recombination products between two or more distinct nucleotide sequences based on any criteria desired by the user. By contrast, natural recombination allows for exchange of nucleotide sequence at equivalent positions along two chromosomes only in regions with substantial homology.

In the method of the invention for creating a collection of recombination products between two or more nucleotide sequences an initial set of oligonucleotides is generated that corresponds to a first nucleotide sequence and one or more subsequent sets of oligonucleotides are generated, each corresponding to a distinct subsequent nucleotide sequence. The initial and subsequent sets of oligonucleotides can be generated such that the entire plus and minus strands of, for example, a gene encoding a polypeptide of interest are represented. The initial and subsequent nucleotide sequences each can encode a distinct amino acid sequence and can have dissimiliar nucleotide sequences, for example, a sequence identity of less than 90%, less than 80%, less than 70%, less than 60%, less than 50%, less than 40%, less than 30%, less than 20%, less than 10%. Furthermore, a set of combination oligonucleotides is generated, where each oligonucleotide contains sequences from the two or more nucleotide sequences corresponding to the first and subsequent sets of oligonucleotides.

Methods for synthesizing oligonucleotides are well known in the art and found in, for example, Oligonucleotide Synthesis: A Practical Approach, Gate, ed., IRL Press, Oxford (1984), which is incorporated herein by reference in its entirety. Additional methods of forming large arrays of oligonucleotides and other polymer sequences in a short period of time have been devised and are described by Pirrung et al., U.S. Pat. No. 5,143,854; Fodor et al., WO 92/10092; and Winkler et al., U.S. Pat. No. 6,136,269, each of which is incorporated herein by reference.

Synthesis of oligonucleotides can be accomplished using both solution phase and solid phase methods. Solid phase oligonucleotide synthesis employs mononucleoside phosphoramidite coupling units and involves reiteratively performing four steps: deprotection, coupling, capping, and oxidation as has been described, for example, by Beaucage and Caruthers, Tetrahedron Letters 22: 1859-1862 (1981), which is incorporated herein by reference. Typically, a first nucleoside, having protecting groups on any exocyclic amine functionalities present, is attached to an appropriate solid support, such as a polymer support or controlled pore glass beads. Activated phosphorus compounds, typically nucleotide phosphoramidites, also bearing appropriate protecting groups, are added step-wise to elongate the growing oligonucleotide, thus 4 forming an oligonucleotide that is bound to a solid support. Once synthesis of the desired length and sequence of oligonucleotide is achieved the oligonucleotide can be deblocked, deprotected and removed from the solid support. The synthesized oligonucleotides can be lyophilized, resuspended in water and 5′ phosphorylated with polynucleotide kinase and ATP to enable ligation. If desired, the phosphoramidite synthesis can be modified by methods known in the art to miniaturize the reaction size and generate small reaction volumes and yields in the range between 1 to 5 nmoles.

Oligonucleotide synthesis via solution phase can be accomplished with several coupling mechanisms, and can include, for example, the use of phosphorous to prepare thymidine dinucleoside and thymidine dinucleotide phosphorodithioates. Methods useful for preparing oligonucleotides via solution phase are well known in the art and described by Sekine et. al., J. Org. Chem. 44:2325 (1979); Dahl, Sulfer Reports, 11:167-192 (1991); Kresse et al., Nucleic Acids Res. 2:1-9 (1975); Eckstein, Ann. Rev. Biochem., 54:367-402 (1985); and Yau, U.S. Pat. No. 5,210,264, each of which is incorporated herein by reference.

An exemplary method for preparing an a set of oligonucleotides involves computer-directed synthesis of nucleic acids as described, for example, in WO 99/14318 A1. The methods of the invention can be accomplished by direct synthesis of nucleotide sequences and design of polypeptides using DNA as a programming tool. For example, a collection of polynucleotide recombination products can be designed and a set of oligonucleotides that correspond to the polynucleotide recombination products can be synthesized, assembled and transferred to a host for expression of the encoded polypeptide. In particular, the initial and subsequent nucleotide sequences, which can encode distinct polypeptides, and the corresponding set of combination oligonucleotides can be designed by computer, virtually converted into sets of parsed oligonucleotides covering the plus and minus strands of the nucleotide sequence and synthesized for subsequent assembly using, for example, the triplet mixing algorithm, to create a collection of polynucleotide recombination products between the two or more nucleotide sequences.

In one embodiment of the invention, a first nucleotide sequence can be selected that encodes a polypeptide of interest and a second nucleotide sequence can be selected that encodes a distinct polypeptide with similar function and dissimilar sequence, with the goal of creating a collection of recombination products, which can be single recombination products, double recombination products or multiple recombination products. Using computer-directed synthesis, a set of combination oligonucleotides can be designed that contains sequence corresponding to each of the first and second nucleotide sequence.

A set of combination oligonucleotides can be designed that contains sequences corresponding to distinct nucleotide sequences, where the permutation or order of sequences on the combination oligonucleotide is designed as desired by the user. For example, a set of combination oligonucleotides can be designed, where each oligonucleotide contains a 5′ region or portion corresponding to the first nucleotide sequence and a 3′ region or portion corresponding to the second nucleotide sequence or vice versa. Alternatively, a set of combination oligonucleotides can be designed, where each oligonucleotide contains regions corresponding to distinct first, second and, if desired, subsequent nucleotide sequences in any order or permutation desired by the user. A set of combination oligonucleotides can be designed to encompass every possible combination of two or more distinct nucleotide sequences or can contain a subset of combinations between the two or more nucleotide sequences, depending on the desired collection of recombination products.

Thus, the resulting collection of recombinant products between two or more nucleotide sequences can be designed as desired by the user. For example, a cognate pair of polypeptides can be selected to create variants based on criteria including, for example, similarity of primary, secondary or tertiary structure, functional similarity or evolutionary ancestry, to encompass single or multiple recombination products of the encoding nucleotide sequences such that the collection of recombination products scans the entire length of the encoding nucleotide sequences with regard to location of the one or more recombination breakpoints. In addition to a cognate pair of polypeptides, where the method would involve a first nucleotide sequence and one subsequent nucleotide sequence, a collection of recombination products also can be created between more than two nucleotide sequences, for example, where it is desirable to create a collection of recombinant products corresponding to a population of polypeptides, for example, a family of related polypeptides or a collection of polypeptides chosen by any criteria desired by the user. For example, amino acid sequences corresponding to unrelated polypeptides can be selected if it is desired to create a collection of polypeptide variants that possess a combination of properties corresponding to each of the unrelated polypeptides.

In addition to scanning the entire length of the distinct nucleotide sequences with regard to the location of the recombination breakpoint, a collection of recombination products can consist of recombination products in one or more predetermined regions of the nucleotide sequence if directed or targeted diversity of recombination products is desired. The regions to be targeted for creating a collection of recombination products can be selected based on the nucleotide sequences or based on the encoded amino acid sequences and further can be selected based on any of the criteria set forth herein or desired by the user. In addition to being targeted, predetermined or all-encompassing, a collection of recombination products can also be prepared so as to reflect recombination events in randomly chosen regions along the sequence.

A set of oligonucleotides can correspond to a nucleotide sequence that is 100, 200, 300, 400, 500, 600, 700, 800, 1000, 1500, 2000, 4000, 8000, 10000, 12000, 18,000, 20,000, 40,000, 80,000 or more nucleotides in length. The initial and subsequent sets of nucleotide sequences encode distinct amino acid sequences, while each member of the set of combination oligonucleotides contains nucleotide sequences corresponding to two or more of the initial and subsequent sets.

In certain embodiments, one initial set, one subsequent set and one set of combination oligonucleotides are generated. However, in other embodiments two or more subsequent sets of oligonucleotides can be generated. Similarly, two or more sets of combination oligonucleotides can be generated, for example, as exemplified herein two sets of combination oligonucleotides corresponding to distinct nucleotide sequences, where one set of combination oligonucleotides has a 5′ region corresponding to the first nucleotide sequence and a 3′ region corresponding to the other nucleotide sequence and where the second set of combination oligonucleotides has the converse configuration are useful to create a collection of polynucleotide recombination products encompassing every possible recombinant between the two sequences.

Computer software can be used to break down the nucleotide sequences into set of overlapping oligonucleotides of specified length to yield a set of oligonucleotides which overlap to cover the particular nucleotide sequence in overlapping sets. In particular, nucleotide sequences can be parsed electronically using a computer algorithm and corresponding executable program which generates sets of overlapping oligonucleotides. For example, a nucleotide sequence of any length, for example, 1000 nucleotides can be broken down into a set of 40 oligonucleotides, each consisting of 50 nucleotides, where 20 members of the set correspond to one strand and the remaining 20 members correspond to the other strand. Alternatively, a nucleotide sequence of any length can be broken down into a set of oligonucleotides having any desired number of components, for example, 100, 90, 80, 70, 60, 50, 40, 30, 20 or less, and each individual oligonucleotide can consist of between about 20 and 100, between about 30 and 90, between about 40 and 80, or between about 50 and 70 nucleotides as described herein. The oligonucleotide members making up the set can be selected to overlap on each strand, for example, by between about 100 and 20 base pairs, between about 90 and 25 base pairs, between about 80 and 30 base pairs, between about 70 and 35 base pairs, or between about 60 and 40 base pairs.

The oligonucleotides can be parsed using, for example, Parseoligo™, a proprietary computer program that optimizes nucleic acid sequence assembly. Optional steps in sequence assembly can include identifying and eliminating sequences that can give rise to hairpins, repeats or other difficult sequences. Additionally, the algorithm can first direct the synthesis of the coding regions to correspond to a desired codon preference, for example, E. coli as shown in Example II for the nucleotide sequences encoding the antibody molecules AF169027 and HAS225092. For conversion of a particular nucleotide sequence encoding a polypeptide to another codon preference, the algorithm utilizes a amino acid sequence to generate a DNA sequence using a specified codon table. Once the nucleotide sequences are broken down into sets of oligonucleotides, chemical synthesis of each of the overlapping sets of oligonucleotides using an array type synthesizer and phosphoamidite chemistry resulting in an array of synthesized oligomers. Thus, a first and one or more subsequent sets of oligonucleotides can be virtually constructed. Similarly, one or more sets of combination oligonucleotides can be constructed that encompass sequences from two or more nucleic acid molecules. Furthermore, as shown in Example II, the sequences to be recombined can be truncated or extended so that they are of equal size.

The design and synthesis of nucleotide sequences encoding distinct amino acid sequences can include the addition of degenerate or mixed bases at specified positions. Degenerate bases are non-canonical bases that exhibit some ability to base pair to any of the 4 standard bases. Exemplary degenerate bases include, for example, “purinel” and “pyrimidine,” which would be the structural scaffolds for A/G and C/T, respectively, as well as fluorine-derivatized bases, and the like. Examples of other degenerate bases include 5-nitroindole, 3-nitropyrrole, and inosine.

Furthermore, the individual oligonucleotides corresponding to the initial and subsequent sets can be designed as multiple distinct sequences so as to increase the diversity of the recombination products that are created. In particular, the diversity of the polynucleotide recombination products can be controlled or directed by targeting of the recombination sites between the nucleotide sequences. Such targeting allows for an increase in the likelihood of productive recombination products that have a desired alteration in bioactivity.

For example, the sites of an encoded polypeptide determined to be important for its bioactivity, for example, the catalytic site of an enzyme or the complementary determining region (CDR) of an antibody, can be targeted in the generation of polynucleotide recombination products. For any polypeptide the information obtained from structural, biochemical and modeling methods can be useful to determine those amino acids predicted to be important for activity. For example, molecular modeling of a substrate in the active site of an enzyme can be utilized to predict amino acid alterations that allow for higher catalytic efficiency based on a better fit between the enzyme and its substrate. Conversely, amino acid alterations of residues important for the functional structure of a polypeptide, which can include intra-chain disulfide bonds, generally are not targeted in the preparation of a collection of polynucleotide recombination products encoding variant polypeptides. It is understood that the functional, structural, or phylogenic features of a polypeptide can be useful to target the site of recombination to create a collection of polynucleotide recombination products with an increased likelihood of possessing a desired characteristic.

As set forth above, the methods of the invention can be practiced to prepare a collection of recombination products between two distinct nucleotide sequences that encode different antibody molecules. The collection of polypeptide variants thus created by the invention method can represent a library of recombination products between different antibody molecules that represent a variety of specific CDR combinations that can subsequently be tested by high throughput screening. Thus, in this embodiment, the invention method enables the preparation of large numbers of synthetic antibodies or antibody-like molecules. As demonstrated in Example II, the recombination of two “single chain” scfv molecules via the invention method can be used to generate a combinatorically large set of antibody variants with novel binding sites and antibody affinities. Although exemplified for two “single chain” antibody molecules where V_{H} and V_{L} binding domains are expressed in single molecule and connected by linker peptide, it is understood that the method of the invention is equally applicable to multiple chain antibody molecules.

The nucleotide sequences further can include non-coding elements such as origins of replication, telomeres, promoters, enhancers, transcription and translation start and stop signals, introns, exon splice sites, chromatin scaffold components and other regulatory sequences. The nucleotide sequences used in the methods of the invention can correspond to prokaryotic or eukaryotic sequences including bacterial, yeast, viral, mammalian, amphibian, reptilian, avian, plants, archebacteria and other DNA containing living organisms.

The oligonucleotide sets can be contain oligonucleotides of between about 10 to 300 or more nucleotide, 15 and 150 nucleotide, between about 20 and 100 nucleotide, between about 25 and 75 nucleotide, between about 30 and 50 nucleotide, or any size in between. Specific lengths include, for example, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64. 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 150 or more nucleotides.

Depending on the size, the overlap between the oligonucleotides of the two strands can be designed to be about 50 percent, about 40 percent, about 30 percent, or about 20 percent of the length of the oligonucleotide or between about 5 and 75 nucleotide per oligonucleotide pair, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64. 65, 66, 67, 68, 69, 70, 71, 72, 73, 74, 75, 80, go, 100 or more nucleotides. The sets can be designed such that complementary pairing results in overlap of paired sequences, as each oligonucleotide of the first strand is complementary with regions from two oligonucleotides of the second strand, with the possible exception of the terminal oligonucleotides. The first and the second strands of oligonucleotides can be annealed in a single mixture and treated with a ligating enzyme.

Either before or after the mixing of the oligonucleotides, but prior to annealing, oligonucleotides can be treated with polynucleotide kinase, for example, T4 polynucleotide kinase. After annealing, the oligonucleotides are treated with an enzyme having a ligating function, for example, a DNA ligase or a topoisomerase, which does not require 5′ phosphorylation.

As set forth herein, the initial and subsequent sets of oligonucleotides, as well as the set of combination oligonucleotides can be generated by computer-directed oligonucleotide synthesis to ultimately result in expression of a collection of recombination products assembled by mixing oligonucleotides from the initial and subsequent sets with the one or more sets of combination oligonucleotides. Thus, computer-directed assembly can be employed to create a collection of polynucleotide recombination products according to the invention method for introduction into host cells and subsequent expression.

A set of oligonucleotides corresponding to a nucleotide sequence can be synthesized, for example, by first selecting two or more amino acid sequences and subsequently generating a parsed set of oligonucleotides covering the plus and minus, also referred to as the forward and reverse, strands of the sequence. A computer program, stored on a computer-readable medium, can be used for generating a nucleotide sequence derived from a model sequence. A computer program also can be used to parse the nucleotide sequences into sets of multiply distinct, partially complementary oligonucleotides corresponding to an initial set, a subsequent set and a set of combination oligonucleotides, and control assembly of the collection of polynucleotide recombination products by controlling the extension of the initiating oligonucleotides of each polynucleotide recombinant by addition of partially complementary oligonucleotides resulting in a collection of contiguous recombination products.

For every polynucleotide recombinant an initiating oligonucleotide can be selected that serves as the first or starting sequence that is extended by addition of a next most terminal oligonucleotide or a next most terminal component polynucleotide. If desired, the addition of a next terminal oligonucleotide can occur so as to sequentially extend the growing polynucleotide. An initiating oligonucleotide can correspond to the initial or a subsequent set of oligonucleotides or can be a combination oligonucleotide and can have a 5′ overhang, a 3′ overhang, or a 5′ and a 3′ overhang of either strand. An initiating oligonucleotide can be extended in an alternating bi-directional manner, in a uni-directional manner or any combination thereof. An initiating oligonucleotide contained in a recombinant of the invention sequence can be either the 5′ most terminal oligonucleotide, the 3′ most terminal oligonucleotide, or neither the 3′ nor the 5′ most terminal nucleotide of the recombinant sequence, depending on whether the recombinant is assembled starting from the middle or whether it is assembled starting from one of the two ends. If an initiating oligonucleotide contained in a recombinant sequence represents either the 5′ most terminal oligonucleotide, the 3′ most terminal oligonucleotide of the target polynucleotide, it can encompass one overhang.

For ligation assembly of a recombinant, an initiating oligonucleotide begins assembly by providing an anchor for hybridization of further oligonucleotides contiguous with the initiating oligonucleotide. As with the initiating oligonucleotides, the subsequently added oligonucleotides can correspond to the initial or a subsequent set of oligonucleotides or can be a combination oligonucleotide depending on the particular mixing algorithm desired. Thus, for ligation assembly, an initiating oligonucleotide can be a partially double-stranded nucleic acid thereby providing single-stranded overhangs for annealing of a contiguous, double-stranded recombinant nucleic acid molecule. For primer extension assembly of a recombinant, an initiating oligonucleotide begins assembly by providing a template for hybridization of subsequent oligonucleotides contiguous with the initiating oligonucleotide. Thus, for primer extension assembly, an initiating oligonucleotide can be partially double-stranded or fully double-stranded.

Once the initial and subsequent sets and the set of combination oligonucleotides are parsed by computer, the information can be used to direct the synthesis of arrays of oligonucleotides or synthesis according to any other organized scheme. For example, an array synthesizer can be directed to produce the oligonucleotides as arrays in microtiter plates of, for example, 23, 46, 96, 192, 384 or 1536 wells of parsed oligonucleotides, each capable of assembly of as many component oligonucleotides. The set of arrayed sequences subsequently can be assembled using a mixed pooling strategy that includes a desired mixing scheme or algorithm, for example, triplet mixing. It is understood, however, that the methods of the invention also can be practiced by mixing schemes involving mixing of more than three oligonucleotides such that, rather than triplexes via triplet mixing, for example, five-plexes to ten-plexes or more, ten-plexes to twenty-plexes or more, twenty-plexes to fifty-plexes or more, fifty-plexes to seventy-five-plexes or more, seventy-five-plexes to one-hundred-plexes or more, one-hundred-plexes to one-hundred-and-fifty-plexes or more, one-hundred-and-fifty-plexes to two-hundred-plexes or more of oligonucleotides are generated by mixing the corresponding number of component oligonucleotides.

To assemble recombination products by triplet mixing groups of three oligonucleotides are combined into a primary pool of triplex or triplet intermediates by combining in a primary pool two adjacent oligonucleotides that correspond to a first strand of a double-stranded nucleic acid molecule, with a third oligonucleotide that corresponds to the opposite strand of the nucleic acid molecule and further has a region of sequence complementarity with each of said two adjacent oligonucleotides of the first strand; subsequently combining two or more of the primary pools containing triplex intermediates into a secondary pool; then combining two or more of the secondary pools into a tertiary pool; and finally combining two or more of the tertiary pools into a final pool.

The triplexes of oligonucleotides are initially formed, for example, having 50 nucleotides each and a 25 base pair overlap with a complementary oligonucleotide. Two of the oligonucleotides correspond to one strand and are ligation substrates joined by ligase and the third oligonucleotide is corresponds to the complementary strand and is a stabilizer that brings together the two specific sequences by annealing a part of the final recombination polynucleotide. Following initial pooling and triplex formation, sets of triplexes are systematically joined, ligated and assembled into larger fragments. Each step is mediated by pooling, ligation and thermal cycling to achieve annealing and denaturation. The final step joins assembled pieces into a complete polynucleotide recombinant sequence representing all the fragment in the array.

Once assembly of the oligonucleotide sets has been completed, the oligonucleotides encompassing the plus strands of each of the initial and subsequent sets and the set of combination oligonucleotides are combined where each oligonucleotide is mixed with the oligonucleotides corresponding to the other sets. Similarly, nucleotides encompassing the minus strands of each of the sets also can be combined separately. Next, assembly is carried out using the algorithm of triplet mixing using the two pools of oligonucleotides. Triplet mixing is one variation of an assembly scheme in which a series of smaller polynucleotides is made by ligating 2, 3, 4, 5, 6, or 7 oligonucleotides into one sequence and adding this to another sequence encompassing the same or a similar number of oligonucleotides parts.

As used herein, the term “triplex mixing” refers to an assembly scheme in which the intermediates are prepared by systematic combination of three oligonucleotides to form a triplex consisting of two oligonucleotides corresponding to one strand and a third oligonucleotide corresponding to the opposite strand and having a region of complementary to each of the first two oligonucleotides so as to allow annealing into a triplex structure. Briefly, the assembly of each member of a collection of polynucleotide recombination products by triplet mixing involves generating a first triplet consisting of an oligonucleotide corresponding to the initial set, the subsequent set or the set of combination oligonucleotides; a second oligonucleotide contiguous with the first oligonucleotide that also corresponds to the initial set, the subsequent set or the set of combination oligonucleotides; and an opposite strand oligonucleotide that has contiguous sequence and is at least partially complementary to the first oligonucleotide and also at least partially complementary to the second oligonucleotide. The first and second oligonucleotides, which correspond to the same strand, are subsequently annealed to the opposite strand oligonucleotide to result in a partially double-stranded intermediate including a 5′ overhang and a 3′ overhang. Next, a second intermediate is generated that is contiguous with the first intermediate and also encompasses a first oligonucleotide corresponding to the initial set, the subsequent set or the set of combination oligonucleotides; a second oligonucleotide contiguous with the first oligonucleotide that also corresponds to the initial set, the subsequent set or the set of combination oligonucleotides; and an opposite strand oligonucleotide that has contiguous sequence and is at least partially complementary to the first oligonucleotide and also at least partially complementary to the second oligonucleotide. As with the first intermediate, the first and second oligonucleotides of the second intermediate, which correspond to the same strand, are annealed to the opposite strand oligonucleotide to result in a partially double-stranded intermediate including a 5′ overhang and a 3′ overhang. In the next step, the first intermediate triplet is contacted with the second intermediate under conditions and for such time suitable for annealing so as to result in an extending, contiguous double-stranded polynucleotide, that can be sequentially contacted with additional triplet intermediates through repeated cycles of annealing and ligation to create a polynucleotide recombinant. Alternatively, if possible given the ligation kinetics, the oligonucleotides can be placed in a mixture and ligation be allowed to proceed.

It is understood that the assembly of polynucleotide recombination products can take place in the absence of primer extension and further can occur in any maaner desired by the user, for example, by sequential or systematic addition of single stranded or double stranded intermediates in either a unidirectional or a bi-directional manner. If desired, the mixture of intermediates, for example, triplexes, five-plexes, seven-plexes, nine-plexes or eleven-plexes of oligonucleotides or any other desired combination of oligonucleotides can be contacted with a ligase under conditions suitable for ligation.

Thus, the set of arrayed oligonucleotides in the plate can be assembled using a mixed pooling strategy. For example, systematic pooling of component oligonucleotides can be performed using a modified Beckman Biomek automated pipetting robot, or another automated lab workstation and the fragments can be combined with buffer and enzyme, for example, Taq I DNA ligase or Egea Assemblase™ or Egea Zipperase™. After each step of pooling in the microwell plates, the temperature can be ramped to enable annealing and ligation, then additional pooling carried out. The systematic pooling of the component oligonucleotides as described herein can be accomplished by methods known in the art, including use of an automated system or workstation.

It is understood that annealing conditions can be adjusted based on the particular strategy used for annealing, the size and composition of the oligonucleotides, and the extent of overlap between the oligonucleotides of the initial and subsequent sets. For example, where all the oligonucleotides are mixed together prior to annealing, heating the mixture to 80° C., followed by slow annealing for between 1 to 12 h is conducted. In the assembly methods of the invention, slow annealing by generally no more than 1.5° C. per minute to 37° C. or below can performed to maximize the efficiency of hybridization. Slow annealing can be accomplished by a variety of methods, for example, with a programmable thermocycler. The cooling rate can be linear or non-linear and can be, for example, 0.1° C., 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C., or 2.0° C. Annealing can be conducted for about 2, about 3, about 4, about 5, about 6, about 7, about 8, about 9, or about 10 h. However, in other embodiments, the annealing time can be as long as 24 h. The cooling rate can be adjusted up or down to maximize efficiency and accuracy.

With the aid of a computer, synthesis of a gene combination using a high throughput oligonucleotide synthesizer as a set of overlapping component oligonucleotides. As described above, the oligonucleotides are assembled using a robotic combinatoric assembly strategy and the assembly ligated using DNA ligase or topoisomerase, followed by transformation into a suitable host strain.

The invention method for the creation of a collection of recombination products between two or more nucleotide sequences, can further comprise the step of amplifying the collection of polynucleotide recombination products.

Processes for amplifying a desired target polynucleotide are known and have been described in the literature. K. Kleppe et al, J. Mol. Biol. 56: 341-361 (1971), disclose a method for the amplification of a desired DNA sequence. The method involves denaturation of a DNA duplex to form single strands. The denaturation step is carried out in the presence of a sufficiently large excess of two nucleic acid primers that hybridize to regions adjacent to the desired DNA sequence. Upon cooling two structures are obtained each containing the full length of the template strand appropriately complexed with primer. DNA polymerase and a sufficient amount of each required nucleoside triphosphate are added whereby two molecules of the original duplex are obtained. The above cycle of denaturation, primer addition and extension are repeated until the appropriate number of copies of the desired target polynucleotide is obtained.

One method of amplification is the polymerase chain reaction (PCR) that involves template-dependent extension using thermally stable DNA polymerase as described by Mullis, Cold Sprinqs Harbor Symp. Ouant. Biol. 51:263-273 (1986); Erlich et al., EP 50,424; EP 84,796; EP 258,017; EP 237,362; Mullis, EP 201,184; Mullis et al, U.S. Pat. No. 4,683,202; Erlich, U.S. Pat. No. 4,582,788; and Saiki et al., U.S. Pat. No. 4,683,194, each of which is incorporated herein by reference. PCR achieves the amplification of a specific nucleotide sequence using two oligonucleotide primers complementary to regions of the sequence to be amplified. Extension products incorporating primers then become templates for subsequent amplification steps. Reviews of the PCR technique are provided by Mullis, supra, 1986; Saki et al., Bio/Technology 3:1008-1012 (1985); and Mullis, Meth. Ensemble. 155:335-350 (1987), each of which is incorporated herein by reference. Thus, a collection of polynucleotide recombination products can be amplified using the polymerase chain reaction and specific primers and, optionally, purified by gel electrophoresis. Either PCR or reverse-transcription PCR (RT-PCR) can be used to produce a polynucleotide recombinant having any desired nucleotide boundaries. Desired modifications to the nucleotide sequence can also be introduced by choosing an appropriate primer with one or more additions, deletions or substitutions. Such nucleotide sequences can be amplified exponentially starting from as little as a single polynucleotide recombination product.

Thus, one method of amplifying a collection of polynucleotide recombination products involves PCR. However, other methods known in the art for amplification of nucleotide sequences also are applicable to the methods of the invention, for example, the ligase chain reaction (LCR), self-sustained sequence replication (3SR), beta replicase, for example, Q-beta replicase, reaction, phage terminal binding protein reaction, strand displacement amplification (SEA) or NASA also can be used to amplify nucleotide sequences (Tipper et al., J. Viral. Heat. 3:267 (1996); Holler et al., Lab. Invest. 73:577 (1995); Yagi et al., Proc. Natl. Acad. Sci. USA 93:5395 (1996); Blanco et al., Proc. Natl. Acad. Sci. USA 91:12198 (1994); Spears et al., Anal. Biochem. 247:130 (1997); Spurge et al., Mol. Cell. Probes 10:247 (1996); Gibbers et al., J. Viol. Methods 66:293 (1997); Edendale et al., Int. J. Food Microbial. 37:13 (1997); and Leone et al., J. Viol. Methods 66:19 (1997)), each of which is incorporated herein by reference. Other polynucleotide amplification procedures can be used and include amplification systems as described by KWh et al., Proc. Natl. Acad. Sci. U.S.A. 86:1173 (1989)); Ginger et al., PCT WO 88/10315; Miller et al., PCT WO 89/06700; Daley et al., EP 329,822; Kramer et al., U.S. Pat. No. 4,786,600; and Wu et al., Genomic 4:560 (1989).

The ligase chain reaction (“LCR”), disclosed in EPO 320, 308, is incorporated herein by reference in its entirety. In LCR, two complementary probe pairs are prepared, and in the presence of a target sequence, each pair will bind to opposite complementary strands of the target such that they abut. In the presence of a ligase, the two probe pairs will link to form a single unit. By temperature cycling, bound ligated units dissociate from the target and then serve as “target sequences” for ligation of excess probe pairs.

For expression of a collection of polynucleotide recombination products between two or more nucleotide sequences created by the methods of the invention, for example, bacterial cells the individual recombination products can contain a sequence corresponding to a bacterial origin of replication such as, for example, pBR322, Bluescript or any other commercially available vector. For transfer into eukaryotic cells, a polynucleotide recombinant should contain the origin of replication of a mammalian virus, chromosome or subcellular component such as mitochondria.

For example, oligonucleotides having a length of 50 nucleotides and an overlap of 25 base pairs that correspond to the initial set, one or more subsequent sets and set of combination oligonucleotides, can be synthesized by an oligonucleotide synthesizer, for example, a Genewriter™ or an oligonucleotide array synthesizer (OAS). The plus strand sets of oligonucleotides are each synthesized in a 96-well plate and the minus strand sets are separately synthesized in 96-well microtiter plates. Synthesis can be carried out using phosphoramidite chemistry modified to miniaturize the reaction size and generate small reaction volumes and yields in the range of 2 to 5 nmole. Synthesis is done on controlled pore glass beads (CPGs), and the polynucleotide recombination products are deblocked, deprotected and removed from the beads and subsequently lyophilized, re-suspended in water and 5′ phosphorylated using polynucleotide kinase and ATP to enable ligation.

For transfer of a polynucleotide recombinant into bacterial cells, it should contain the sequence for a bacterial origin of replication, for example, pBR322. Oligonucleotides can be added by ligation chain reaction or any other assembly method adding one or more oligonucleotides at each step. For the performance of a ligase chain reaction, the first oligonucleotide in the chain is attached to a solid support, for example, an agarose bead. The second oligonucletide is added along with DNA ligase, and annealing and ligation reaction carried out, and the beads are washed. The second, overlapping oligonucleotide from the opposite strand is added, annealed and ligation carried out. The third oligonucleotide is added and ligation carried out. This procedure is replicated until all oligonucleotides are added and ligated. This procedure is best carried out for long sequences using an automated device. The DNA sequence is removed from the solid support, a final ligation is carried out, and the molecule transferred into host cells.

As described herein, a set of combination oligonucleotides can be synthesized such that each of the set of combination oligonucleotides contains sequence corresponding to the initial nucleotide sequence and further contains sequence corresponding to at least one of the one or more subsequent nucleotide sequences. For example, in those embodiments involving an initial set of oligonucleotides corresponding to a first nucleotide sequence and one subsequent set of oligonucleotides corresponding to a distinct subsequent nucleotide sequence, where the initial and subsequent nucleotide sequences each encode a distinct amino acid sequence, each of the set of combination oligonucleotides can comprise a 5′ portion corresponding to the first nucleotide sequence and a 3′ portion corresponding to the subsequent nucleotide sequence.

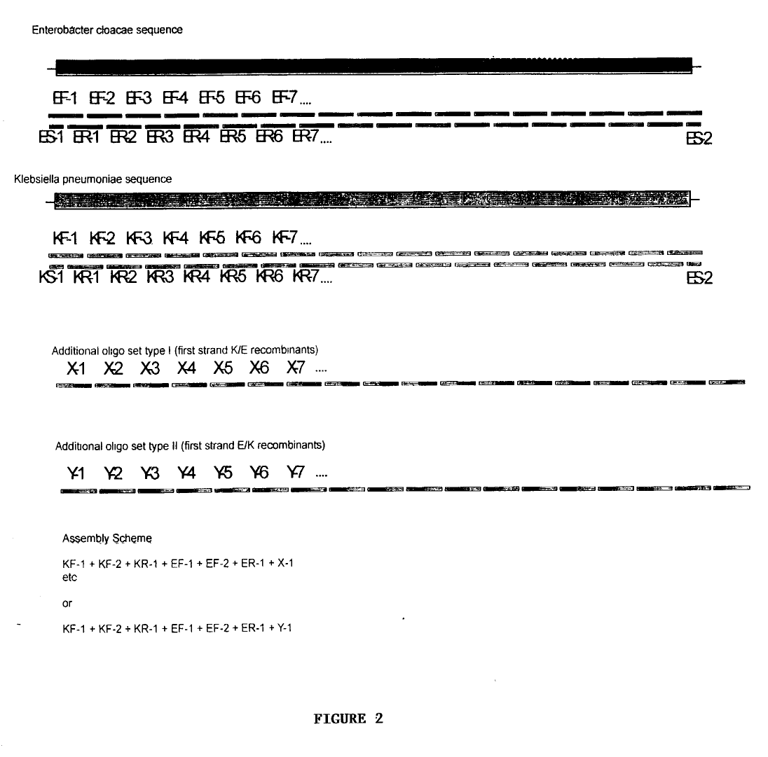

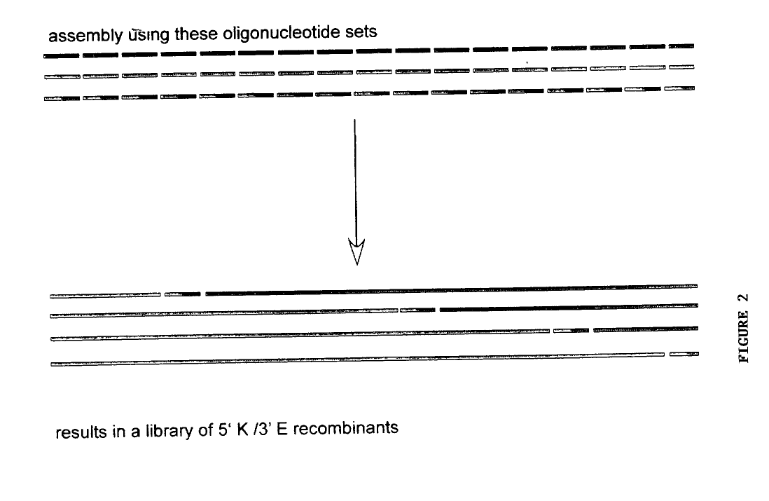

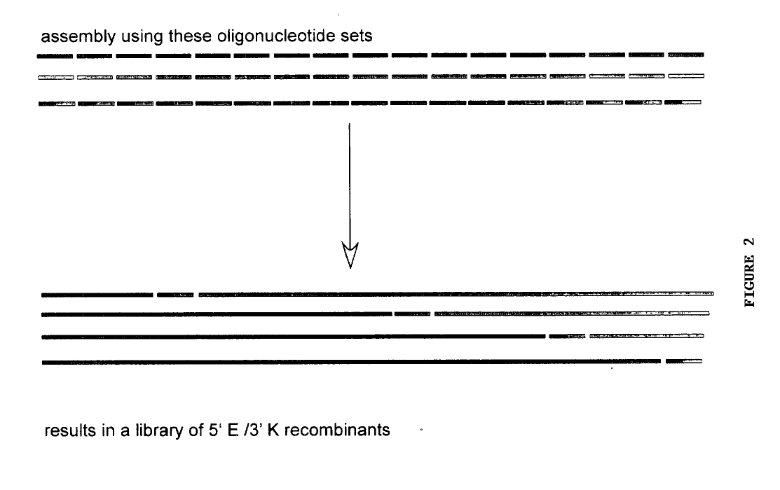

As shown schematically in FIG. 2 and described in Example I, for the beta lactamase sequences of E. Cloacae and K. Pneumonia, carrying out assembly of polynucleotide recombination products using the algorithm of triplet mixing where the combination oligonucleotides comprise a 5′ portion corresponding to E. Cloacae (E) and a 3′ portion corresponding to K. Pneumonia (K) the result is the creation of a collection of every possible single 5′E/3′K polynucleotide recombination products. This exemplification of the invention method demonstrates assembly of a collection of polynucleotide recombinants via one of the embodiments, in which the polynucleotide recombinants are assembled by combining an initial set of oligonucleotides, one subsequent set of oligonucleotides and one combination set of oligonucleotides. Conversely, in a related embodiment, an initial set of oligonucleotides corresponding to a first nucleotide sequence and one subsequent set of oligonucleotides corresponding to a distinct subsequent nucleotide sequence, where the initial and subsequent nucleotide sequences each encode a distinct amino acid sequence, each of the set of combination oligonucleotides can comprise a 3′ portion corresponding to the first nucleotide sequence and a 5′ portion corresponding to the subsequent nucleotide sequence. As shown in FIG. 2 and described in Example I, for the beta lactamase sequences of E. Cloacae and K. Pneumonia, carrying out assembly of polynucleotide recombination products using the algorithm of triplet mixing where the combination oligonucleotides comprise a 3′ portion corresponding to E. Cloacae (E) and a 3′ portion corresponding to K. Pneumonia (K), the result is the creation of a collection of every possible single 3′E/5′K polynucleotide recombination products.

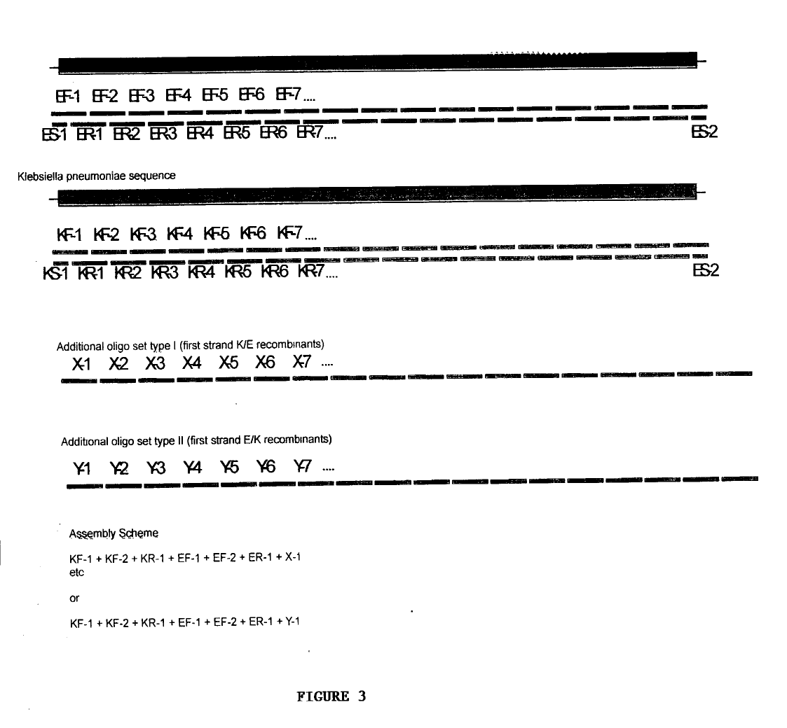

To create a collection of polynucleotide recombination products that contains every possible single and multiple recombinant, two sets of combination oligonucleotides can be generated, where one of the sets of combination oligonucleotides consists of oligonucleotides a 3′ portion corresponding to a first nucleotide sequence and a 5′ portion corresponding to a subsequent nucleotide sequence and where the second set of the combination oligonucleotides consists of oligonucleotides encompassing a 3′ portion corresponding to the subsequent nucleotide sequence and a 5′ portion corresponding to the first nucleotide sequence. As shown schematically in FIG. 3, for the beta lactamase sequences of E. Cloacae and K. Pneumonia, carrying out assembly of polynucleotide recombination products using the algorithm of triplet mixing where one set of combination oligonucleotides consists of oligonucleotides encompassing a 3′ portion corresponding to E. Cloacae (E) and a 3′ portion corresponding to K. Pneumonia (K), and a second set of combination oligonucleotides consists of oligonucleotides encompassing a 5′ portion corresponding to E. Cloacae (E) and a 3′ portion corresponding to K. Pneumonia (K), the result is the creation of a collection of every possible single and multiple recombinant.

Thus, in a particular embodiment, the invention provides a method of creating a collection of recombination products between two genes including (a) selecting a first and a second amino acid sequence; (b) generating a first set of oligonucleotides corresponding to a first nucleotide sequence and a second set of oligonucleotides corresponding to a second nucleotide sequence, where the first and second nucleotide sequences correspond to the first and second amino acid sequences, and where the first and the second nucleotide sequences each consist of a plus and a minus strand; (c) generating a set of combination oligonucleotides, each of the set of combination oligonucleotides encompassing sequence corresponding to the plus strand of the first nucleotide sequence and encompassing sequence corresponding to the plus strand of the second nucleotide sequence; (d) preparing a first oligonucleotide pool including the plus strand corresponding to the first nucleotide sequence, the plus strand corresponding to the second nucleotide sequence and the set of combination oligonucleotides; (e) preparing a second oligonucleotide pool including the minus strands corresponding to the first and second nucleotide sequences; and (f) assembling a collection of recombination products by triplet mixing using the first and the second oligonucleotide pool.

It is understood that modifications which do not substantially affect the activity of the various embodiments of this invention also are included within the definition of the invention provided herein. The following examples are intended to illustrate but not limit the present invention.

### EXAMPLE I

### Creation of Beta-Lactamase Recombination Products from K. Pneumoniae and E. Cloacae

This example describes the creation of a collection of recombination products between two beta-lactamase polypeptides that have similar structures and dissimilar sequences.

The K. Pneumoniae and E. Cloacae beta lactamase proteins consist of 286 amino acids encoded by 858 bases and 292 amino acids encoded by 886 bases, respectively, and are 31.1% identical. To construct a collection of recombination products between the two polypeptides, two sets of oligonucleotides, the first set corresponding to the K. Pneumoniae beta-lactamase and the subsequent set corresponding to the E. Cloacae beta lactamase, are designed and synthesized that each consisted of thirty-six 50-mers, 18 corresponding to each strand. There are two spacer oligonucleotides, one on each end, to create terminal blunt ends. These are called “S” oligonucleotides, with Sl denoting the 5′ end and S2 denoting the 3′ end. Oligonucleotides on the forward strand are denoted “F” followed by a number, ranging from Fl to Fn depending on the number of oligonucleoties. Similarly, oligonucleotides on the reverse strand are denoted “R” followed by a number, ranging from R1 to R(n-1). In addition, a third set of combination oligonucleotides is synthesized, each of which contains the 5′ 25 bases from K. Pneumoniae, the 3′ 25 bases from E. Cloacae and represents the plus strand.

Following the design and synthesis, the first and subsequent sets of plus strand oligonucleotides corresponding to K. Pneumoniae and E. Cloacae, respectively, and the recombinant set are combined and mixed as shown in FIG. 2. Similarly, the first and subsequent sets of minus strand oligonucleotides are combined and mixed as shown in FIG. 2.

Assembly of the recombination products is subsequently carried out utilizing the algorithm of triplet mixing of the combined set of plus strand oligonucleotides and the combined set of minus strand oligonucleotides. Briefly, the oligonucleotides are combined into pools, each pool having primarily three oligonucleotides. Each pool of three oligonucleotides is set up to contain two adjacent oligonucleotides on one strand, and a single oligonucleotide on the other strand, which is complementary to a 25 bp stretch on each of the other two oligonucleotides. Using a robotic liquid handling system such as for example, the Packard Multiprobe II, the oligonucleotides are transferred from stock plates into a reaction vessel, for example, a PCR plate or tubes, creating a series of primary pools. Each primary pool contains the appropriate oligonucleotides, as well as 40 units of Taq ligase and the appropriate buffer. The final volume is 50 ml. The reaction tubes are placed in a thermal cycler at 80° C. for 5 minutes, followed by 15 minutes at 70° C.

The primary pools are subsequently combined to form secondary pools, with each secondary pool containing 25 ml of either two or three primary pools. The reaction tubes are placed into a thermal cycler for the above cited conditions. The secondary pools are then combined to form tertiary pools, with each tertiary pool containing either two or three secondary pools. The reaction tubes are placed into a thermal cycler for the above cited conditions.

To create a final pool, 25 ml each of two, three or four tertiary pools are combined. The reaction tubes are placed into a thermal cycler for the above cited conditions. After the final thermal cycling step, the reaction products are purified over a Qiagen PCR spin column to remove single oligonucleotides and small, incomplete hybridization products. Varying amounts, including 1 ml, 2 ml, and 5 ml, of the purified assembly reaction is PCR amplified using a universal set of primers that flank the gene using standard conditions and visualized on an ethidium bromide stained agarose gel. The PCR reactions with the strongest, cleanest band and least background is then cloned into a suitable vector, used to transform E. Coli cells and selected on ampicillin plates.

The result of this construction is a group of ampicillin resistant colonies expressing beta-lactamase that consists of all possible mixed recombination products, such that the 5′portion always corresponds to K. Pneumoniae and the 3′portion always corresponds to E. Cloacae.

Alternatively, to generate a library of recombination products where the 3′portion always corresponds to K. Pneumoniae and the 5′portion always corresponds to E. Cloacae, the third set of combination oligonucleotides is simply synthesized so that each contains the 3′ 25 bases from K. Pneumoniae, the 5′ 25 bases from E. Cloacae and represents the plus strand.

Furthermore, to generate a library of all possible single and multiple recombination products both sets of combination oligonucleotides are used as shown in FIG. 3, one set where the 5′portion always corresponds to K. Pneumoniae and the 3′portion always corresponds to E. Cloacae, the other set of combination oligonucleotides where the 3′ portion 25 bases from K. Pneumoniae, the 5′ 25 bases from E. Cloacae and represents the plus strand. Since there are 18 oligonucleotide positions and four possibilities at each position the resulting collection of recombination products will have 4¹⁸ distinct sequences.

### EXAMPLE II

### Creation of New Antibody Binding Sites through Recombination of two Dissimilar Variable Chain Regions

This example describes the creation of a collection of polypeptide variants corresponding to synthetic antibody molecules formed by recombination between two antibodies of known antigenic specificity and dissimilar sequence.

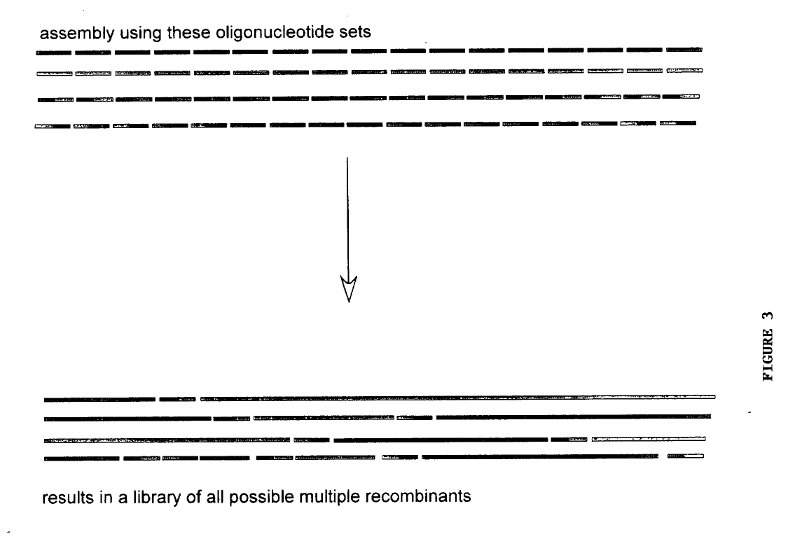

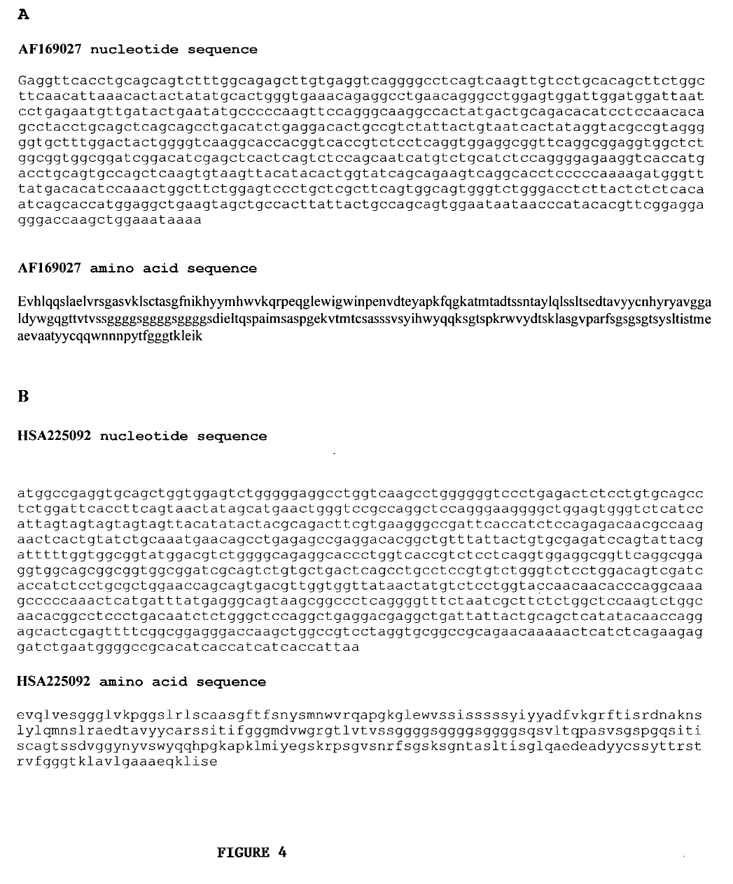

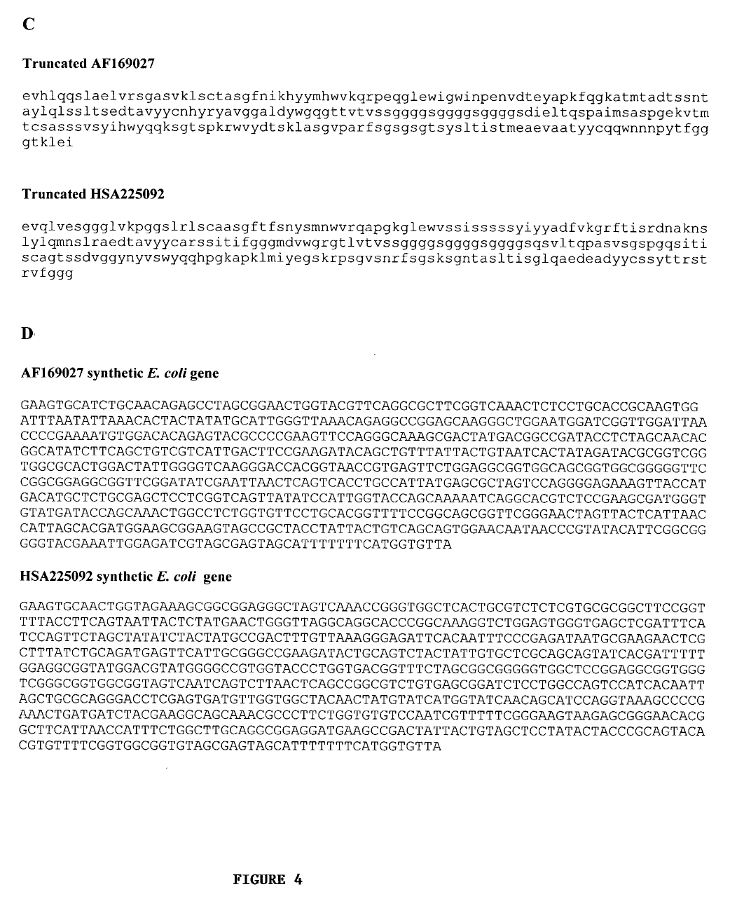

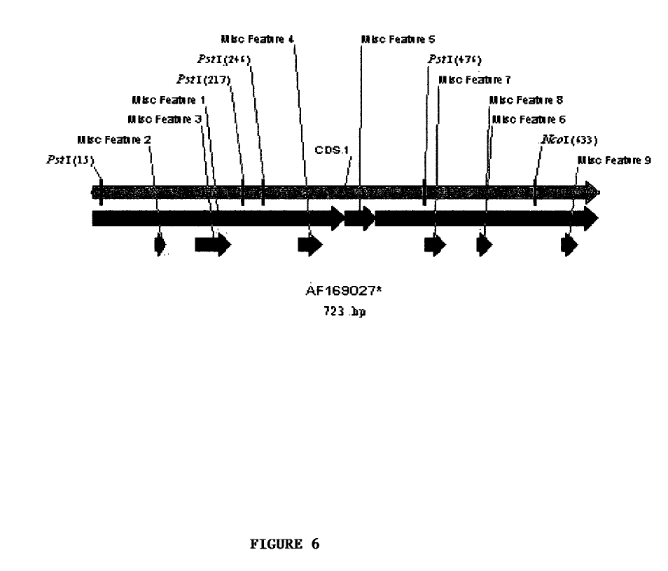

AF169027 is a single chain mouse monoclonal antibody shown in FIG. 6 that combines a V_{H} and V_{L} chain with a peptide linker. Each V_{H} or V_{L} has three CDR regions, also known as also known as hypervariable regions, containing a portion of the binding site and the majority of variability in sequence. As shown in FIG. 4(A), the nucleotide sequence of AF169027 is 723 base pairs and corresponds to a protein of 241 amino acids.

HSA225092 is a human single chain antibody of unspecified reactivity. As shown in FIG. 4(B), the nucleotide sequence of HSA225092 is 819 base pairs defining a protein of 257 amino acids. The sequence identity is 46.1% between the two peptide chains. This level of similarity is probably not sufficient to allow recombination to occur in living cells.

Prior to recombination of the initial and subsequent nucleotide sequences, each of the corresponding amino acid sequences is shortened by truncation to make two sequences of equal length, 240 amino acids, as shown in FIG. 4(C).

Subsequently, the synthetic genes shown in FIG. 4(D) are derived based on E.coli codon preferences. Each synthetic gene is synthesized using 50-mer oligonucleotides and adding padding sequences at each end to make the entire construct 750 bp.

The following initial set of oligonucleotides is used for assembling the AF169027 synthetic E. coli gene:

| | |
|---|---|
| AF-F-1 | |
| 5GAAGTGCATCTGCAACAGAGCCTAGCGGAACTGGTACGTTCAGGCGCTTC | [SEQ ID NO:11] |
| AF-F-2 | |
| 5GGTCAAACTCTCCTGCACCGCAAGTGGATTTAATATTAAACACTACTATA | [SEQ ID NO:12] |
| AF-F-3 | |
| 5 TGCATTGGGTTAACAGAGGCCGGAGCAAGGGCTGGATGGATCGGTTGG | [SEQ ID NO:13] |
| AF-F-4 | |
| 5ATTAACCCCGAAAATGTGGACACAGAGTACGCCCCGAAGTTCCAGGGCAA | [SEQ ID NO:14] |
| AF-F-5 | |
| 5AGCGACTATGACGGCCGATACCTCTAGCAACACGGCATATCTTCAGCTGT | [SEQ ID NO:15] |
| AF-F-6 | |
| 5CGTCATTGACTTCCGAAGATACAGCTGTTTATTACTGTAATCACTATAGA | [SEQ ID NO:16] |
| AF-F-7 | |
| 5TACGCGGTCGGTGGCGCACTGGACTATTGGGGTCAAGGGACCACGGTAAC | [SEQ ID NO:17] |
| AF-F-8 | |
| 5CGTGAGTTCTGGAGGCGGTGGCAGCGGTGGCGGGGGTTCCGGCGGAGGCG | [SEQ ID NO:18] |
| AF-F-9 | |
| 5GTTCGGATATCGAATTAACTCAGTCACCTGCCATTATGAGCGCTAGTCCA | [SEQ ID NO:19] |
| AF-F-10 | |
| 5GGGGAGAAAGTTACCATGACATGCTCTGCGAGCTCCTCGGTCAGTTATAT | [SEQ ID NO:20] |
| AF-F-11 | |
| 5CCATTGGTACCAGCAAAAATCAGGCACGTCTCCGAAGCGATGGGTGTATG | [SEQ ID NO:21] |
| AF-F-12 | |
| 5ATACCAGCAAACTGGCCTCTGGTGTTCCTGCACGGTTTTCCGGCAGCGGT | [SEQ ID NO:22] |
| AF-F-13 | |
| 5TCGGGAACTAGTTACTCATTAACCATTAGCACGATGGAAGCGGAAGTAGC | [SEQ ID NO:23] |
| AF-F-14 | |
| 5CGCTACCTATTACTGTCAGCAGTGGAACAATAACCCGTATACATTCGGCG | [SEQ ID NO:24] |
| AF-F-15 | |
| 5GGGGTACGAAATTGGAGATCGTAGCGAGTAGCATTTTTTTCATGGTGTTA | [SEQ ID NO:25] |
| AF-S-1 | |
| 5CTAGGCTCTGTTGCAGATGCACTTC | [SEQ ID NO:26] |
| AF-R-1 | |
| 5ACTTGCGGTGCAGGAGAGTTTGACCGAAGCGCCTGAACGTACCAGTTCCG | [SEQ ID NO:27] |
| AF-R-2 | |
| 5TCCGGCCTCTGTTTAACCCAATGCATATAGTAGTGTTTAATATTAAATCC | [SEQ ID NO:28] |
| AF-R-3 | |
| 5CTGTGTCCACATTTTCGGGGTTAATCCAACCGATCCATTCCAGCCCTTGC | [SEQ ID NO:29] |
| AF-R-4 | |
| 5AGAGGTATCGGCCGTCATACTCGCTTTGCCCTGGAACTTCGGGGCGTACT | [SEQ ID NO:30] |
| AF-R-5 | |
| 5GCTGTATCTTCGGAAGTCAATGACGACAGCTGAAGATATGccGTGTTGcT | [SEQ ID NO:31] |
| AF-R-6 | |
| 5AGTCCAGTGCGCCACCGACCGCGTATCTATAGTGATTACAGTAATAAACA | [SEQ ID NO:32] |
| AF-R-7 | |
| 5GCTGCCACCGCCTCCAGAACTCACGGTTACCGTGGTCCCTTGACCCCAAT | [SEQ ID NO:33] |
| AF-R-8 | |
| 5GACTGAGTTAATTCGATATCCGAACCGCCTCCGCCGGAACCCCCGCCACC | [SEQ ID NO:34] |
| AF-R-9 | |
| 5AGCATGTCATGGTAACTTTCTCCCCTGGACTAGCGCTCATAATGGCAGGT | [SEQ ID NO:35] |
| AF-R-10 | |
| 5GCCTGATTTTTGCTGGTACCAATGGATATAACTGACCGAGGAGCTCGCAG | [SEQ ID NO:36] |
| AF-R-11 | |
| 5ACACCAGAGGCCAGTTTGCTGGTATCATACACCCATCGCTTCGGAGACGT | [SEQ ID NO:37] |
| AF-R-12 | |
| 5TGGTTAATGAGTAACTAGTTCCCGAACCGCTGCCGGAAAACCGTGCAGGA | [SEQ ID NO:38] |
| AF-R-13 | |
| 5CCACTGCTGACAGTAATAGGTAGCGGCTACTTCCGCTTCCATCGTGCTAA | [SEQ ID NO:39] |
| AF-R-14 | |
| 5GCTACGATCTCCAATTTCGTACCCCCGCCGAATGTATACGCGTTATTGTT | [SEQ ID NO:40] |
| AF-S-2 | |
| 5TAACACCATGAAAAAAATGCTACTC | [SEQ ID NO:41] |

The following subsequent set of oligonucleotides is used for assembling the HSA225092 synthetic E. coli gene [SEQ ID NO:42]:

| | |
|---|---|
| HS-F-1 | |
| 5GAAGTGCAACTGGTAGAAAGCGGCGGAGGGCTAGTCAAACCGGGTGGCTC | [SEQ ID NO:43] |
| HS-F-2 | |
| 5ACTGCGTCTCTCGTGCGCGGCTTCCGGTTTTACCTTCAGTAATTACTCTA | [SEQ ID NO:44] |
| HS-F-3 | |
| 5TGAACTGGGTTAGGCAGGCACCCGGCAAAGGTCTGGAGTGGGTGAGCTCG | [SEQ ID NO:45] |
| HS-F-4 | |
| 5ATTTCATCCAGTTCTAGCTATATCTACTATGCCGACTTTGTTAAAGGGAG | [SEQ ID NO:46] |
| HS-F-5 | |
| 5ATTCACAATTTCCCGAGATATGCGAAGAACTCGCTTTATCTGCAGATGA | [SEQ ID NO:47] |
| HS-F-6 | |
| 5GTTCATTGCGGGCCGAAGATACTGCAGTCTACTATTGTGCTCGCAGCAGT | [SEQ ID NO:48] |
| HS-F-7 | |
| 5ATCACGATTTTTGGAGGCGGTATGGACGTATGGGGCCGTGGTACCCTGGT | [SEQ ID NO:49] |
| HS-F-8 | |
| 5GACGGTTTCTAGCGGCGGGGGTGGCTCCGGAGGCGGTGGGTCGGGCGGTG | [SEQ ID NO:50] |
| HS-F-9 | |
| 5GCGGTAGTCAATCAGTCTTAACTCAGCCGGCGTCTGTGAGCGGATCTCCT | [SEQ ID NO:51] |
| HS-F-10 | |
| 5GGCCAGTCCATCACAATTAGCTGCGCAGGGACCTCGAGTGATGTTGGTGG | [SEQ ID NO:52] |
| HS-F-11 | |
| 5CTACAACTATGTATCATGGTATCAACAGCATCCAGGTAAAGCCCCGAAC | [SEQ ID NO:53] |
| HS-F-12 | |
| 5TGATGATCTACGAAGGCAGCAAACGCCCTTCTGGTGTGTCCAATCGTTTT | [SEQ ID NO:54] |
| HS-F-13 | |
| 5TCGGGAAGTAAGAGCGGGAACACGGCTTCATTAACCATTTCTGGCTTGCA | [SEQ ID NO:55] |
| HS-F-14 | |
| 5GGCGGAGGATGAAGCCGACTATTACTGTAGCTCCTATACTACCCGCAGTA | [SEQ ID NO:56] |
| HS-F-15 | |
| 5CACGTGTTTTCGGTGGCGGTGTAGCGAGTAGCATTTTTTTCATGGTGTTA | [SEQ ID NO:57] |
| HS-S-16 | |
| 5CGCCGCTTTCTACCAGTTGCACTTC | [SEQ ID NO:58] |
| HS-R-1 | |
| 5GGAAGCCGCGCACGAGAGACGCAGTGAGCCACCCGGTTTGACTAGCCCTC | [SEQ ID NO:59] |
| HS-R-2 | |
| 5CCGGGTGCCTCCCTAACCCAGTTCATAGAGTAATTACTGAAGCTAAAACC | [SEQ ID NO:60] |
| HS-R-3 | |
| 5AGATATAGCTAGAACTGGATGAAATCCAGCTCACCCACTCCAGACCTTTG | [SEQ ID NO:61] |
| HS-R-4 | |
| 5CGCATTATCTCGGGAAATTGTGAATCTCCCTTTAACAAAGTCGGCATAGT | [SEQ ID NO:62] |
| HS-R-5 | |
| 5GCAGTATCTTCGGCCCGCAATGAACTCATCTGCAGATAAAGCGAGTTCTT | [SEQ ID NO:63] |
| HS-R-6 | |
| 5CCATACCGCCTCCAAAAATCGTGATACTGCTGCGAGCACAATAGTAGACT | [SEQ ID NO:64] |
| HS-R-7 | |
| 5GCCACCCCCGCCGCTAGAAACCGTCACCAGGGTACCACGGCCCCATACGT | [SEQ ID NO:65] |
| HS-R-8 | |
| 5TGAGTTAAGACTGATTGACTACCGCCACCGCCCGACCCACCGCCTCCGGA | [SEQ ID NO:66] |
| HS-R-9 | |
| 5CGCAGCTAATTGTGATGGACTGGCCAGGAGATCCGCTCACAGACGCCGGC | [SEQ ID NO:67] |
| HS-R-10 | |
| 5TTGATACCATGATACATAGTTGTAGCCACCAACATCACTCGAGGTCCCTG | [SEQ ID NO:68] |
| HS-R-11 | |
| 5CGTTTGCTGCCTTCGTAGATCATCAGTTTCGGGGCTTTACCTGGATGCTG | [SEQ ID NO:69] |
| HS-R-12 | |
| 5CCGTGTTCCCGCTCTTACTTCCCGAAAAACGATTGGACACACCAGAAGGG | [SEQ ID NO:70] |
| HS-R-13 | |
| 5GTAATAGTCGGCTTCATCCTCCGCCTGCAAGCCAGAATGGTTAATGAAG | [SEQ ID NO:71] |
| HS-R-14 | |
| 5GCTACACCGCCACCGAAAACACGTGTACTGCGGGTAGTATAGGAGCTACA | [SEQ ID NO:72] |
| HS-S-2 | |
| 5TAACACCATGAAAAAAATGCTACTC | [SEQ ID NO:73] |

The assembly of these sequences using the methods of the invention generates the native form of each antibody protein.

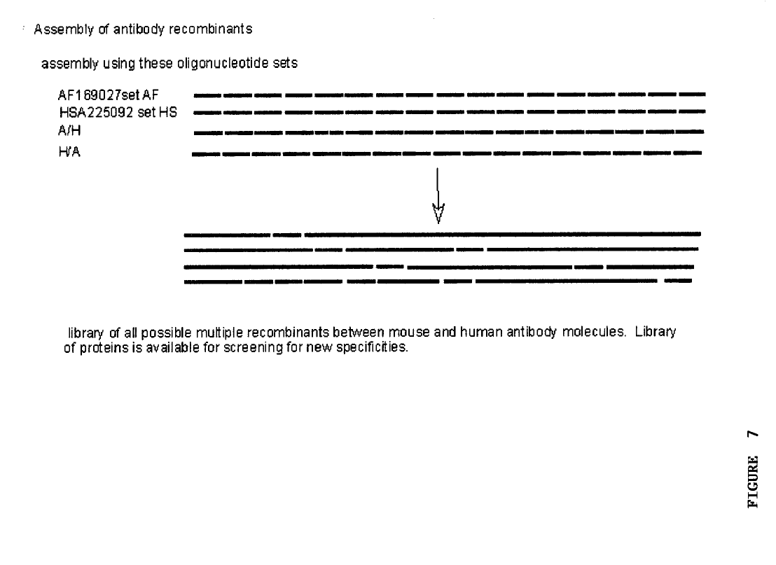

In addition, a third set of combination oligonucleotides is synthesized each of which contains the 5′ 25 bases from AF169027 and the 3′ 25 bases from HSA225092 and represents the plus strand. Following the design and synthesis, the initial, subsequent and combination sets of oligonucleotides are combined as schematically shown in FIG. 7 to produce a collection of recombination products that correspond to antibody polypeptide variants. These synthetic antibodies can be be screened for additional or novel binding activities. The combination set of oligonucleotides (A/H):

| | |
|---|---|
| A/HF-F-1 | |
| 5GAAGTGCATCTGCAACAGAGCCTAGGAGGGCTAGTCAAACCGGGTGGCTC | [SEQ ID NO:74] |
| A/HF-F-2 | |
| 5CGTCAAACTCTCCTGCACCGCAAGTGGTTTTACCTTCAGTAATTACTCTA | [SEQ ID NO:75] |
| A/HF-F-3 | |
| 5TGCATTGGGTTAAACAGAGGCCGGACAAAGGTCTGGAGTGGGTGAGCTCG | [SEQ ID NO:76] |
| A/HF-F-4 | |
| 5ATTAACCCCGAAAATGTGGACACAGACTATGCCGACTTTGTTAAAGGGAG | [SEQ ID NO:77] |
| A/HF-F-5 | |
| 5AGCGACTATGACGGCCGATACCTCTAAGAACTCGCTTTATCTGCAGATGA | [SEQ ID NO:78] |
| A/HF-F-6 | |
| 5CGTCATTGACTTCCGAAGATACAGCAGTCTACTATTGTGCTCGCAGCAGT | [SEQ ID NO:79] |
| A/HF-F-7 | |
| 5TACGCGGTCGGTGGCGCACTGGACTACGTATGGGGCCGTGGTACCCTGGT | [SEQ ID NO:80] |
| A/HF-F-8 | |
| 5CGTGAGTTCTGGAGGCGGTGGCAGCTCCGGAGGCGGTGGGTCGGGCGGTG | [SEQ ID NO:81] |
| A/HF-F-9 | |
| 5GTTCGGATATCGAATTAACTCAGTCGCCGGCGTCTGTGAGCGGATCTCCT | [SEQ ID NO:82] |
| A/HF-F-10 | |
| 5GGGGAGAAAGTTACCATGACATGCTCAGGGACCTCGAGTGATGTTGGTGG | [SEQ ID NO:83] |
| A/HF-F-11 | |
| 5CCATTGGTACCAGCAAAAATCAGGCCAGCATCCAGGTAAAGCCCCGAAAC | [SEQ ID NO:84] |
| A/HF-F-12 | |
| 5ATACCAGCAAACTGGCCTCTGGTGTCCCTTCTGGTGTGTCCAATCGTTTT | [SEQ ID NO:85] |
| A/HF-F-13 | |
| 5TCGGGAACTAGTTACTCATTAACCACTTCATTAACCATTTCTGGCTTGCA | [SEQ ID NO:86] |
| A/HF-F-14 | |
| 5CGCTACCTATTACTGTCAGCAGTGGTGTAGCTCCTATACTACCCGCAGTA | [SEQ ID NO:87] |
| A/HF-F-15 | |
| 5GGGGTACGAAATTGGAGATCGTAGCGAGTAGCATTTTTTTCATGGTGTTA | [SEQ ID NO:88] |

Similarly, a second set of combination oligonucleotides is synthesized where the 5′ 25 bases are from HSA225092 and the 3′ 25 bases are from AF169027. Assembly of this set with the initial and subsequent sets generates a set of all recombinantion products where the 5′ portion is HSA225092 and the 3′ portion is AF169027.

| | |
|---|---|
| H/AF-F-1 | |
| 5GAAGTGCAACTGGTAGAAAGCGGCGCGGAACTGGTACGTTCAGGCGCTTC | [SEQ ID NO:89] |
| H/AF-F-2 | |
| 5ACTGCGTCTCTCGTGCGCGGCTTCCGGATTTAATATTAAACACTACTATA | [SEQ ID NO:90] |
| H/AF-F-3 | |
| 5TGAACTGGGTTAGGCAGGCACCCGGGCAAGGGCTGGAATGGATCGGTTGG | [SEQ ID NO:91] |
| H/AF-F-4 | |
| 5ATTTCATCCAGTTCTAGCTATATCTAGTACGCCCCGAAGTTCCAGGGCAA | [SEQ ID NO:92] |
| H/AF-F-5 | |
| 5ATTCACAATTTCCCGAGATAATGCGAGCAACACGGCATATCTTCAGCTGT | [SEQ ID NO:93] |
| H/AF-F-6 | |
| 5GTTCATTGCGGGCCGAAGATACTGCTGTTTATTACTGTAATCACTATAGA | [SEQ ID NO:94] |
| H/AF-F-7 | |
| 5ATCACGATTTTTGGAGGCGGTATGGATTGGGGTCAAGGGACCACGGTAAC | [SEQ ID NO:95] |
| H/AF-F-8 | |
| 5GACGGTTTCTAGCGGCGGGGGTGGCGGTGGCGGGGGTTCCGGCGGAGGCG | [SEQ ID NO:96] |
| H/AF-F-9 | |
| 5GCGGTAGTCAATCAGTCTTAACTCAACCTGCCATTATGAGCGCTAGTCCA | [SEQ ID NO:97] |
| H/AF-F-10 | |
| 5GGCCAGTCCATCACAATTAGCTGCGCTGCGAGCTCCTCGGTCAGTTATAT | [SEQ ID NO:98] |
| H/AF-F-11 | |
| 5CTACAACTATGTATCATGGTATCAAACGTCTCCGAAGCGATGGGTGTATG | [SEQ ID NO:99] |
| H/AF-F-12 | |
| 5TGATGATCTACGAAGGCAGCAAACGTCCTGCACGCTTTTCCGGCAGCGGT | [SEQ ID NO:100] |
| H/AF-F-13 | |
| 5TCGGGAAGTAAGAGCGGGAACACGGTTAGCACGATGGAAGCGGAAGTAGC | [SEQ ID NO:101] |
| H/AF-F-14 | |
| 5GGCGGAGGATGAAGCCGACTATTACAACAATAACCCGTATACATTCGGCG | [SEQ ID NO:102] |
| H/AF-F-15 | |
| 5CACGTGTTTTCGGTGGCGGTGTAGCGAGTAGCATTTTTTTCATGGTGTTA | [SEQ ID NO:103] |

Similarly, assembly using all four sets, which is the intial, subsequent and two sets of combination oligonucleotides, generates a collection of recombinantion products that represent all possible multiple recombinations between AF169027 and HSA225092.

### EXAMPLE III

### Creation of Recombinants Between Lipocalin Binding Domains

This example describes the creation of a collection of recombination products between two lipocalin polypeptides that have similar structures and dissimilar sequences

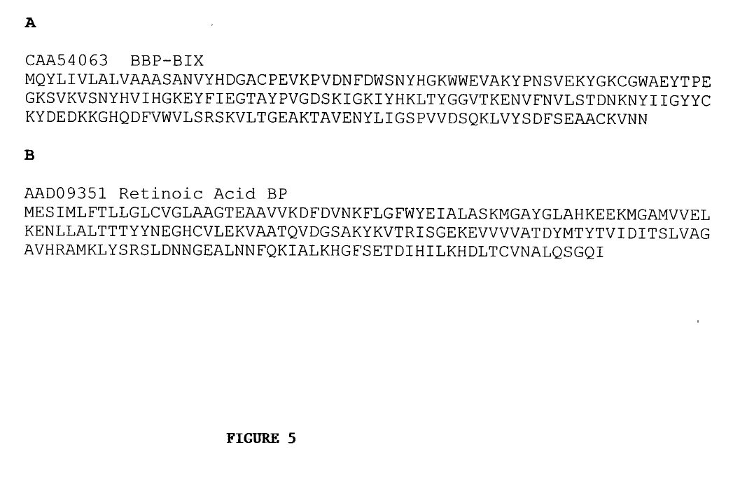

BBP-B1X is the biliverdin binding protein of a butterfly species, the amino acid sequence of which is shown in FIG. 5(A). Retinoic binding protein is a human protein responsible for binding retinoic acid, the amino acid sequence of which is shown in FIG. 5(B).

An initial set of oligonucelotides is prepared that corresponds to the BBP-BIX nucleotide sequence [SEQ ID NO:104]

| | |
|---|---|
| 24 mer | |
| TTTTTTTTTTTTTTTTTTTTTTTT | [SEQ ID NO:106] |
| 48 mer | |
| TTTTTTTTTTTTTTTTTTTTTTTTTTTTTTTTTTTTTTTTTTTTTTTT | [SEQ ID NO:107] |
| 50 merA | |
| ATGCAGCTGGCACGACAGGTATGCAGCTGGCACGACAGGTATGCAGCTGA | [SEQ ID NO:108] |
| 50 merG | |
| ATGCAGCTGGCACGACAGGTATGCAGCTGGCACGACAGGTATGCAGCTGG | [SEQ ID NO:109] |
| 50 merT | |
| ATGCAGCTGGCACGACAGGTATGCAGCTGGCACGACAGGTATGCAGCTGT | [SEQ ID NO:110] |
| 50 merC | |
| ATGCAGCTGGCACGACAGGTATGCAGCTGGCACGACAGGTATGCAGCTGC | [SEQ ID NO:111] |
| BBP-BIX-F-1 | |
| 5GAAAGCGGATGTTGCGGGTTGTTGTTCTGCGGGTTCTGTTCTTCGTTGAC | [SEQ ID NO:112] |
| BBP-BIX-F-2 | |
| 5ATGAGGTTGCCCCGTATTCAGGAATTCTGTTTGGAAACTGTCATGCAGTA | [SEQ ID NO:113] |
| BBP-BIX-F-3 | |
| 5CCTGATCGTTCTGGCGCTGGTTGCGGCGGCGTCTGCGAACGTTTACCACG | [SEQ ID NO:114] |
| BBP-BIX-F-4 | |
| 5ACGGTGCGTGCCCGOAAGTTAAACCGGTTGACAACTTCGACTGGTCTAAC | [SEQ ID NO:115] |
| BBP-BIX-F-5 | |
| 5TACCACGGTAAATGGTGGGAAGTTGCGAAATACCCGAACTCTGTTGAAAA | [SEQ ID NO:116] |
| BBP-BIX-F-6 | |
| 5ATACGGTAAATGCGGTTGGGCGGAATACACCCCGGAAGGTAAATCTGTTA | [SEQ ID NO:117] |
| BBP-BIX-F-7 | |
| 5AAGTTTCTAACTACCACGTTATCCACGGTAAAGAATACTTCATCGAAGGT | [SEQ ID NO:118] |
| BBP-BIX-F-8 | |
| 5ACCGCGTACCCGGTTGGTGACTCTAAAATCGGTAAAATCTACCACAAACT | [SEQ ID NO:119] |
| BBP-BIX-F-9 | |
| 5GACCTACGGTGGTGTTACCAAAGAAAACGTTTTCAACGTTCTGTCTACCG | [SEQ ID NO:120] |
| BBP-BIX-F-10 | |
| 5ACAACAAAAACTACATCATCGGTTACTACTGCAAATACGACGAAGACAAA | [SEQ ID NO:121] |
| BBP-BIX-F-11 | |
| 5AAAGGTCACCAGGACTTCGTTTGGGTTCTGTCTCGTTCTAAAGTTCTGAC | [SEQ ID NO:122] |
| BBP-BIX-F-12 | |
| 5CGGTGAAGCGAAAACCGCGGTTGAAAACTACCTGATCGGTTCTCCGGTTG | [SEQ ID NO:123] |
| BBP-BIX-F-13 | |
| 5TTGACTCTCAGAAACTGGTTTACTCTGACTTCTCTGAAGCGGCCTCCAAA | [SEQ ID NO:124] |
| BBP-BIX-F-14 | |
| 5GTTAACAACACTCTCATACCATGGAAGCTTGCAGTAGCGAGTAOCATTTT | [SEQ ID NO:125] |
| BBP-BIX-F-15 | |
| 5TTTCATGGTGTTATTCCCGATGCTTTTTGAAGTTCGCAGAATCGTATGTG | [SEQ ID NO:126] |
| BBP-BIX-S-1 | |
| 5ACAACAACCCGCAACATCCGCTTTC | [SEQ ID NO:127] |
| BBP-BIX-R-1 | |
| 5ATTCCTGAATACGGGGCAACCTCATGTCAACGAAGAACAGAACCCGCAGA | [SEQ ID NO:128] |
| BBP-BIX-R-2 | |
| 5CGCAACCAGCGCCAGAACGATCAGGTACTGCATGACAGTTTCCAAACAGA | [SEQ ID NO:129] |
| BBP-BIX-R-3 | |
| 5GGTTTAACTTCCGGGCACGCACCGTCGTGGTAAACGTTCGCAOACGCCCC | [SEQ ID NO:130] |
| BBP-BIX-R-4 | |
| 5CAACTTCCCACCATTTACCGTGGTAGTTAGACCAGTCGAAGTTGTCAACC | [SEQ ID NO:131] |
| BBP-BIX-R-5 | |
| 5TTCCGCCCAACCGCATTTACCGTATTTTTCAACAGAGTTCGGGTATTTCG | [SEQ ID NO:132] |
| BBP-BIX-R-G | |
| 5TGGATAACGTGGTAGTTAGAAACTTTAACAGATTTACCTTCCGGGGTGTA | [SEQ ID NO:133] |
| BBP-BIX-R-7 | |
| 5TAGAGTCACCAACCGGGTACGCGGTACCTTCGATGAAGTATTCTTTACCG | [SEQ ID NO:134] |
| BBP-BIX-R-8 | |
| 5TTCTTTGGTAACACCACCGTAGGTCAGTTTGTGGTAGATTTTACCGATTT | [SEQ ID NO:135] |
| BBP-BIX-R-9 | |
| 5TAACCGATGATGTAGTTTTTGTTGTCGGTAGACAGAACGTTGAAAACGTT | [SEQ ID NO:136] |
| BBP-BIX-R-10 | |
| 5CCCAAACGAAGTCCTGGTGACCTTTTTTGTCTTCGTCGTATTTGCAGTAG | (SEQ ID NO:137] |
| BBP-BIX-R-11 | |
| 5TTCAACCGCGGTTTTCGCTTCACCGGTCAGAACTTTAGAACGAGACAGAA | [SEQ ID NO:138] |
| BBP-BTX-R-12 | |
| 5GAGTAAACCAGTTTCTGAGAGTCAACAACCGGAGAACCGATCAGGTAGTT | [SEQ ID NO:139] |
| BBP-BIX-R-13 | |
| 5TCCATGGTATGAGAGTGTTGTTAACTTTGCACGCCGCTTCAGAGAAGTCA | [SEQ ID NO:140] |
| BBP-BIX-R-14 | |
| 5AAGCATCGGGAATAACACCATGAAAAAAATGCTACTCGCTACTGCAAGCT | [SEQ ID NO:141] |
| BBP-BIX-S-2 | |
| 5CACATACGATTCTGCGAACTTCAAA | [SEQ ID NO:142] |

A subsequent set of oligonucleotides corresponding to the Retinoic Acid Binding Protein (RA BP) nucleotide sequence also is prepared:

| | |
|---|---|
| 24 mer | |
| TTTTTTTTTTTTTTTTTTTTTTTT | [SEQ ID NO:106] |
| 48 mer | |
| TTTTTTTTTTTTTTTTTTTTTTTTTTTTTTTTTTTTTTTTTTTTTTTT | [SEQ ID NO:107] |
| 50 merA | |
| ATGCAGCTGGCACGACAGGTATGCAGCTGGCACGACAGGTATGCAGCTGA | [SEQ ID NO:108] |
| 50 merG | |
| ATGCAGCTGGCACGACAGGTATGCAGCTGGCACGACAGGTATGCAGCTGG | [SEQ ID NO:109] |
| 50 merT | |
| ATGCAGCTGGCACGACAGGTATGCAGCTGGCACGACAGGTATGCAGCTGT | [SEQ ID NO:110] |
| 50 merC | |
| ATGCAGCTGGCACGACAGGTATGCAGCTGGCACGACAGGTATGCAGCTGC | [SEQ ID NO:lll] |
| RA BP-F-1 | |
| 5GGTTAGGAAAGCGGATGTTGCGGGTTGTTGTTCTGCGGGTTCTGTTCTTC | [SEQ ID NO:143] |
| RA BP-F-2 | |
| 5GTTGACATGAGGTTGCCCCGTATTCAGGAATTCTGTTTGGAAACTGTCAT | [SEQ ID NO:144] |
| PA BP-F-3 | |
| 5GGAATCTATCATGCTGTTCACCCTGCTGGGTCTGTGCGTTGGTCTGGCGG | [SEQ ID NO:145] |
| PA BP-F-4 | |
| 5CGGGTACCGAAGCGGCGGTTGTTAAAGACTTCGACGTTAACAAATTCCTG | [SEQ ID NO:146] |
| PA BP-F-5 | |
| 5GGTTTCTGGTACGAAATCGCGCTGGCGTCTAAAATGGGTGCGTACGGTCT | [SEQ ID NO:147] |
| PA BP-E-6 | |
| 5GGCGCACAAAGAAGAAAAAATGGGTGCGATGGTTGTTGAACTGAAAGAAA | [SEQ ID NO:148] |
| PA BP-F-7 | |
| 5ACCTGCTGGCGCTGACCACCACCTACTACAACGAAGGTCACTGCGTTCTG | [SEQ ID NO:149] |
| PA BP-F-8 | |
| 5GAAAAAGTTGCGGCGACCCAGGTTGACGGTTCTGCGAAATACAAAGTTAC | [SEQ ID NO:150] |
| PA BP-E-9 | |
| 5CCGTATCTCTGGTGAAAAAGAAGTTGTTGTTGTTGCGACCGACTACATGA | [SEQ ID NO:151] |
| PA BP-F-10 | |
| 5CCTACACCGTTATCGACATCACCTCTCTGGTTGCGGGTGCGGTTCACCGT | [SEQ ID NO:152] |
| PA BP-F-11 | |
| 5GCGATGAAACTGTACTCTCGTTCTCTGGACAACAACGGTGAAGCGCTGAA | [SEQ ID NO:153] |
| PA BP-F-12 | |
| 5CAACTTCCAGAAAATCGCGCTGAAACACGGTTTCTCTGAAACCGACATCC | [SEQ ID NO:154] |
| PA BP-F-13 | |
| 5ACATCCTGAAACACGACCTGACCTGCGTTAACGCGCTGCAGTCTGGTCAG | [SEQ ID NO:155] |
| PA BP-F-14 | |
| 5ATCACTCTCATACCATGGAAGCTTGCAGTAGCGAGTAGCATTTTTTTCAT | [SEQ ID NO:156] |
| PA BE-F-15 | |
| 5GGTGTTATTCCCGATGCTTTTTGAAGTTCGCAGAATCGTATGTGTAGAAA | [SEQ ID NO:157] |
| PA BE-S-1 | |
| 5ACCCGCAACATCCGCTTTCCTAACC | [SEQ ID NO:158] |
| PA BE-R-1 | |
| 5GAATACGGGGCAACCTCATGTCAACGAAGAACAGAACCCGCAGAACAACA | [SEQ ID NO:159] |
| PA BP-R-2 | |
| 5CAGGGTGAACAGCATGATAGATTCCATGACAGTTTCCAAACAGAATTCCT | [SEQ ID NO:160] |
| PA BE-R-3 | |
| 5TTAACAACCGCCGCTTCGGTACCCGCCGCCAGACCAACGCACAGACCCAG | [SEQ ID NO:161] |
| PA BE-R-4 | |
| 5CCAGCGCGATTTCGTACCAGAAACCCAGGAATTTGTTAACGTCGAAGTCT | [SEQ ID NO:162] |
| PA BP-R-5 | |
| 5ACCCATTTTTTCTTCTTTGTGCGCCAGACCGTACGCACCCATTTTAGACG | [SEQ ID NO:163] |
| PA BE-R-6 | |
| 5TAGGTGGTGGTCAGCGCCAGCAGGTTTTCTTTCAGTTCAACAACCATCGC | [SEQ ID NO:164] |
| PA BP-R-7 | |
| 5CAACCTGGGTCGCCGCAACTTTTTCCAGAACGCAGTGACCTTCGTTGTAG | [SEQ ID NO:165] |
| PA BP-R-8 | |
| 5AACTTCTTTTTCACCAGAGATACGGGTAACTTTGTATTTCGCAGAACCGT | [SEQ ID NO:166] |
| PA BP-R-9 | |
| 5GAGGTGATGTCGATAACGGTGTAGGTCATGTAGTCGGTCGCAACAACAAC | [SEQ ID NO:167] |
| PA BP-R-10 | |
| 5GAGAACGAGAGTACAGTTTCATCGCACGGTGAACCGCACCCGCAACCAGA | [SEQ ID NO:168] |
| PA BP-R-11 | |
| 5TTTCAGCGCGATTTTCTGGAAGTTGTTCAGCGCTTCACCGTTGTTGTCCA | [SEQ ID NO:169] |
| PA BP-R-12 | |
| 5CAGGTCAGGTCGTGTTTCAGGATGTGGATGTCGGTTTCAGAGAAACCGTG | [SEQ ID NO:170] |
| PA BP-R-13 | |
| 5CAAGCTTCCATGGTATGAGAGTGATCTGACCAGACTGCAGCGCGTTAACG | [SEQ ID NO:171] |
| PA BP-R-14 | |
| 5TTCAAAAAGCATCGGGAATAACACCATGAAAAAAATGCTACTCGCTACTG | [SEQ ID NO:172] |
| PA BP-S-2 | |
| 5TTTCTACACATACGATTCTGCGAAC | [SEQ ID NO:173] |

Using the initial and subsequent sets of oligonucletides set forth above, each of the native genes can be assembled. Following this, specific collections of recombination products can be generated using the following set of combination oligonucleotides, where the 5′ 25 bases comes from BBP and the 3′ 25 bases from RA BP:

| | |
|---|---|
| BBP-BIX_RA-F-1 | |
| 5GAAAGCGGATCTTGCGGGTTGTTGTTGTTGTTCTGCGGGTTCTGTTCTTC | [SEQ ID NO:174] |
| BBP-EIX_RA-F-2 | |
| 5ATGAGGTTGCCCCGTATTCAGGAATAGGAATTCTGTTTGGAAACTGTCAT | [SEQ ID NO:175] |
| BBP-BIX RA-F-3 | |
| 5CCTGATCGTTCTGGCGCTGGTTGCGCTGGGTCTGTGCGTTGGTCTGGCGG | [SEQ ID NO:176] |
| BBP-BIX RA-F-4 | |
| 5ACGGTGCGTGCCCGGAAGTTAAACCAGACTTCGACGTTAACAAATTCCTG | [SEQ ID NO:177] |
| BBP-BIX RA-F-5 | |
| 5TACCACGGTAAATGGTGGGAAGTTGCGTCTAAAATGGGTGCGTACGGTCT | [SEQ ID NO:178] |
| BBP-BIX RA-F-6 | |
| 5ATACGGTAAATGCGGTTGGGCGGAAGCGATGGTTGTTGAACTGAAAGAAA | [SEQ ID NO:179] |
| BEP-BIX RA-F-7 | |
| 5AAGTTTCTAACTACCACGTTATCCACTACAACGAAGGTCACTGCGTTCTG | [SEQ ID NO:180] |
| BBP-BIX RA-F-8 | |
| 5ACCGCGTACCCGGTTGGTGACTCTAACGGTTCTGCGAAATACAAAGTTAC | [SEQ ID NO:181] |
| BBP-BIX RA-F-9 | |
| 5CACCTACGGTGGTGTTACCAAAGAAGTTGTTGTTGCGACCGACTACATGA | [SEQ ID NO:182] |
| BEP-BIX RA-F-10 | |
| 5ACAACAAAAACTACATCATCGGTTATCTGGTTGCGGGTGCGGTTCACCGT | [SEQ ID NO:183] |
| BBP-BIX RA-F-11 | |
| 5AAAGGTCACCAGGACTTCGTTTGGGTGGACAACAACGGTCAAGCGCTGAA | [SEQ ID NO:184] |
| BBP-BIX RA-F-12 | |
| 5CGGTGAAGCGAAAACCGCGGTTGAACACGGTTTCTCTGAAACCGACATCC | [SEQ ID NO:185] |
| BBP-BIX RA-F-13 | |
| 5TTGACTCTCAGAAACTGGTTTACTCCCTTAACGCGCTCCAGTCTGGTCAG | [SEQ ID NO:186] |
| BBP-BIX RA-F-14 | |
| 5GTTAACAACACTCTCATACCATGGACAGTAGCGAGTAGCATTTTTTTCAT | [SEQ ID NO:187] |
| BBP-BIX RA-F-15 | |
| 5TTTCATGGTGTTATTCCCGATGCTTGTTCGCAGAATCGTATGTGTAGAAA | [SEQ ID NO:188] |
| BEP-BIX RA-R-1 | |
| 5ATTCCTGAATACGGGGCAACCTCATGAAGAACAGAACCCGCAGAACAACA | [SEQ ID NO:189] |
| BBP-BTX RA-R-2 | |
| 5CGCAACCAGCGCCAGAACGATCAGGATGACAGTTTCCAAACAGAATTCCT | [SEQ ID NO:190] |
| BBP-BTX RA-R-3 | |
| 5GGTTTAACTTCCGGGCACGCACCGTCCGCCAGACCAACGCACAGACCCAG | [SEQ ID NO:191] |
| BEP-BIX RA-R-4 | |
| 5CAACTTCCCACCATTTACCGTGGTACAGGAATTTGTTAACGTCGAAGTCT | [SEQ ID NO:192] |
| BEP-BIX RA-R-5 | |
| 5TTCCGCCCAACCGCATTTACCGTATAGACCGTACGCACCCATTTTAGACG | [SEQ ID NO:193] |
| BBP-BIX RA-R-6 | |
| 5TGGATAACGTGGTAGTTAGAAACTTTTTCTTTCAGTTCAACAACCATCGC | [SEQ ID NO:194] |
| BBP-BIX RA-R-7 | |
| 5TAGAGTCACCAACCGGGTACGCGGTCAGAACGCAGTCACCTTCGTTGTAG | [SEQ ID NO:195] |
| BBP-BIX RA-R-8 | |
| 5TTCTTTGGTAACACCACCGTAGGTCGTAACTTTGTATTTCGCAGAACCGT | [SEQ ID N0:196] |
| BBP-BIX RA-R-9 | |
| 5TAACCGATGATGTAGTTTTTGTTGTTCATGTAGTCGGTCGCAACAACAAC | [SEQ ID NO:197] |
| BBP-BIX RA-R-10 | |
| 5CCCAAACGAAGTCCTGGTGACCTTTACGGTGAACCGCACCCGCAACCAGA | [SEQ ID NO:198] |
| BEP-BIX RA-R-11 | |
| 5TTCAACCGCGGTTTTCGCTTCACCGTTCAGCGCTTCACCGTTGTTGTCCA | [SEQ ID NO:199] |
| BBP-BIX RA-R-12 | |
| 5GAGTAAACCAGTTTCTGAGAGTCAAGGATGTCGGTTTCAGAGAAACCGTG | [SEQ ID NO:200] |
| BBP-BIX RA-R-13 | |
| 5TCCATGGTATGAGAGTGTTGTTAACCTGACCAGACTGCAGCGCGTTAACG | [SEQ ID NO:201] |
| BBP-BIX RA-R-14 | |
| 5AAGCATCGGGAATAACACCATGAAAATGAAAAAAATGCTACTCGCTACTG | [SEQ ID NO:202] |

Similarly, a second set of combination oligonucleotides, where the 5′ portion comes from RA and the 3′ portion from BBP is prepared to generate a complementary set of recombinantion products:

| | |
|---|---|
| RA EBP-BIX-F-1 | |
| 5GGTTAGGAAAGCGGATGTTGCGGGTTCTGCGGGTTCTGTTCTTCGTTGAC | [SEQ ID NO:203] |
| PA BBP-BIX-F-2 | |
| 5GTTGACATGAGGTTGCCCCGTATTCTCTGTTTGGAAACTGTCATGCAGTA | [SEQ ID NO:204] |
| RA BBP-BIX-F-3 | |
| 5GGAATCTATCATGCTGTTCACCCTCGCGGCGTCTGCGAACGTTTACCACG | [SEQ ID NO:205] |
| RA BBP-BIX-P-4 | |
| 5CGGGTACCGAAGCGGCGGTTGTTAAGGTTGACAACTTCGACTGGTCTAAC | [SEQ ID NO:206] |
| RA BBP-BIX-F-5 | |
| 5GGTTTCTGGTACGAAATCGCGCTGGCGAAATACCCGAACTCTGTTGAAAA | [SEQ ID NO:207] |
| PA BBP-BIX-F-6 | |
| 5GGCGCACAAAGAAGAAAAAATGGGTTACACCCCGGAAGGTAAATCTGTTA | [SEQ ID NO:208] |
| PA BBP-BIX-F-7 | |
| 5ACCTGCTGGCGCTGACCACCACCTACGGTAAAGAATACTTCATCGAAGGT | [SEQ ID NO:209] |
| PA BBP-BIX-F-8 | |
| 5GAAAAAGTTGCGGCGACCCAGGTTGAAATCGGTAAAATCTACCACAAACT | [SEQ ID NO:210] |
| PA BBP-BIX-F-9 | |
| 5CCGTATCTCTGGTGAAAAAGAAGTTAACGTTTTCAACGTTCTGTCTACCG | [SEQ ID NO:211] |
| PA BBP-BIX-F-10 | |
| 5CCTACACCGTTATCGACATCACCTCCTACTGCAAATACGACGAAGACAAAA | [SEQ ID NO:212] |
| PA BBP-BIX-F-11 | |
| 5GCGATGAAACTGTACTCTCGTTCTCTTCTGTCTCGTTCTAAAGTTCTGAC | [SEQ ID NO:213] |
| PA BBP-BIX-F-12 | |
| 5CAACTTCCAGAAAATCGCGCTGAAAAACTACCTGATCGGTTCTCCGGTTG | [SEQ ID NO:214] |
| PA BBP-BIX-E-13 | |
| 5ACATCCTGAAACACGACCTGACCTGTGACTTCTCTGAAGCGGCGTGCAAA | [SEQ ID NO:2l5] |
| PA BBP-BIX-F-14 | |
| 5ATCACTCTCATACCATGGAAGCTTGAGCTTGCAGTAGCGAGTAGCATTTT | [SEQ ID NO:216] |
| PA BBP-BIX-F-15 | |
| 5GGTGTTATTCCCGATGCTTTTTGAATTTGAAGTTCGCAGAATCGTATGTG | [SEQ ID NO:217] |
| PA BBP-BIXR1 | |
| 5GAATACGGGGCAACCTCATGTCAACGTCAACGAAGAACAGAACCCGCAGA | [SEQ ID NO:218] |
| PA BBP-BIX-R2 | |
| 5CAGGGTGAACAGCATGATAGATTCCTACTGCATGACAGTTTCCAAACAGA | [SEQ ID NO:219] |
| RA BBP-BIX-R3 | |
| 5TTAACAACCGCCGCTTCGGTACCCGCGTGGTAAACGTTCGCAGACGCCGC | [SEQ ID NO:220] |
| RA BBP-BIX-R-4 | |
| 5CCAGCGCGATTTCGTACCAGAACCGTTAGACCAGTCGGTTGTCAACC | [SEQ ID NO:221] |
| PA BBP-BIX-R-5 | |
| 5ACCCATTTTTTCTTCTTTGTGCGCCTTTTCAACAGAGTTCGGGTATTTCG | [SEQ ID NO:222] |
| PA BBP-BIX-R-6 | |
| 5TAGGTGGTGGTCAGCGCCAGCAGGTTAACAGATTTACCTTCCGGGGTGTA | [SEQ ID NO:223] |
| PA BBP-BIX-R-7 | |
| 5CAACCTGGGTCGCCGCAACTTTTTCACCTTCGATGAAGTATTCTTTACCG | [SEQ ID NO:224] |
| PA BBP-BIX-R-8 | |
| 5AACTTCTTTTTCACCAGAGATACGGAGTTTGTGGTAGATTTTACCGATTT | [SEQ ID NO:225] |
| PA BBP-BIX-R-9 | |
| 5GAGGTGATGTCGATAACGGTGTAGGCGGTAGACAGAACGTTGAAAAACGTT | [SEQ ID NO:226] |
| PA BBP-BIX-R10 | |
| 5GAGAACGAGAGTACAGTTTCATCGCTTTGTCTTCGTCGTATTTGCAGTAG | [SEQ ID NO:227] |
| PA BBP-BIX-R-11 | |
| 5TTTCAGCGCGATTTTCTGGAAGTTGGTCAGAACTTTAGAACGAGACAGAA | [SEQ ID NO:228] |
| PA BBP-BIX-R-12 | |
| 5CAGGTGAGGTCGTGTTTCAGGATGTCAACCGGAGAACCGATCAGGTAGTT | [SEQ ID NO:229] |
| PA BBP-BIX-R-13 | |
| 5CAAGCTTCCATGGTATGAGAGTGATTTTGCACGCCGCTTCAGAGAAGTCA | [SEQ ID NO:230] |
| PA BBP-BIX-R-14 | |
| 5TTCAAAAAGCATCGGGAATAACACCAAAATGCTACTCGCTACTGCAAGCT | [SEQ ID NO:231] |

Carrying out an assembly process using all four sets of oligonucleotides, specifically, the intial set, the subsequent set and the two sets of combination oligonucleotides, generates a set of all possible multiple recombinantion products between the two proteins.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A method of creating a collection of recombination products between two nucleotide sequences comprising combining an initial set of oligonucleotides corresponding to a first nucleotide sequence with a subsequent set of oligonucleotides corresponding to a distinct nucleotide sequence and further combining said initial and subsequent sets of oligonucleotides with one or more sets of combination oligonucleotides, each of said combination oligonucleotides comprising a sequence region corresponding to said initial nucleotide sequence and a sequence region corresponding to said second oligonucleotide sequence.

2. A method of creating a collection of recombination products between two or more nucleotide sequences, said method comprising the steps of:
(a) generating an initial set of oligonucleotides corresponding to a first nucleotide sequence and one or more subsequent sets of oligonucleotides, each of said subsequent sets corresponding to a distinct subsequent nucleotide sequence;
(b) generating one or more sets of combination oligonucleotides, each of said combination oligonucleotides comprising a sequence region corresponding to said initial nucleotide sequence and further comprising a sequence region corresponding to at least one of said one or more subsequent nucleotide sequences; and
(c) assembling a collection of polynucleotide recombination products by combining oligonucleotides corresponding to each of said sets.

3. The method of claim 1 or 2, further comprising amplification of said recombination products.

4. The method of claim 1 or 2, wherein said initial and said subsequent nucleotide sequences each encode a distinct amino acid sequence.

5. The method of claim 1 or 2, wherein said collection of recombination products is expressed to obtain a corresponding collection of polypeptide variants.

6. The method of claim 1 or 2, wherein said polypeptide variants represent a collection of synthetic antibody molecules.

7. The method of claim 1 or 2, wherein said oligonucleotides corresponding to each of said sets are combined by triplet mixing of oligonucleotides, said triplet mixing comprising the steps of:
(a) combining groups of three oligonucleotides into a primary pool, wherein two fo said oligonucleotides are adjacent and correspond to a first strand of a double-stranded nucleic acid moelcule, and wherein a third oligonucleotide corresponds to the opposite strand of said double-stranded nucleic acid molecule and further has a region of sequence complementarity with each of said two adjacent oligonucleotides of said first strand;
(b) combining two or more of said primary pools into a secondary pool;
(c) combining two or more of said secondary pools into a tertiary pool; and
(d) combining two or more of said tertiary pools into a final pool.

8. The method of claims 1 or 2, wherein one set of combination oligonucleotides is generated.

9. The method of claim 8, wherein each of said combination oligonucleotides comprises a 3′ portion corresponding to a sequence region of said first nucleotide sequence and a 5′ portion corresponding to a sequence region of said subsequent nucleotide sequence.

10. The method of claim 8, wherein each of said combination oligonucleotides comprises a 3′ portion corresponding to a sequence region of said subsequent nucleotide sequence and a 5′ portion corresponding to a sequence region of said initial nucleotide sequence.

11. The method of claim 9 or 10, wherein said collection consists of single recombination products.

12. The method of claim 1 or 2, wherein two sets of combination oligonucleotides are generated.

13. The method of claim 12, wherein one of said sets of combination oligonucleotides consists of oligonucleotides comprising a 3′ portion corresponding to a sequence region of said first nucleotide sequence and a 5′ portion corresponding to a sequence region of said subsequent nucleotide sequence.

14. The method of claim 13, wherein said second set of said combination oligonucleotides consists of oligonucleotides comprising a 3′ portion corresponding to a sequence region of said subsequent nucleotide sequence and a 5′ portion corresponding to a sequence region of said first nucleotide sequence.

15. The method of claim 14, wherein said collection consists of multiple recombination products.

16. The method of claim 1 or 2, wherein said initial and subsequent sets of oligonucleotides each correspond to a plus strand and a minus strand.

17. The method of claim 16, wherein said set of combination oligonucleotides corresponds to plus strand sequences.

18. The method of claim 17, wherein said set of combination oligonucleotides corresponds to minus strand sequences.

19. The method of claim 1 or 2, wherein said initial and subsequent nucleotide sequences have a sequence identity of less than 50 percent.

20. The method of claim 1 or 2, wherein said initial and subsequent nucleotide sequences have a sequence identity of less than 40 percent.

21. The method of claim 1 or 2, wherein each oligonucleotide comprises 50 nucleotides.

22. A method of creating a collection of recombination products between two genes, said method comprising the steps of:
(a) selecting a first and a second amino acid sequence, wherein said first and second amino acid sequences are encoded by distinct genes;
(b) generating a first set of oligonucleotides corresponding to a first nucleotide sequence and a second set of oligonucleotides corresponding to a second nucleotide sequence, wherein said first and second nucleotide sequences correspond to said first and second amino acid sequences, and wherein said first and said second nucleotide sequences each consist of a plus and a minus strand;
(c) generating a set of combination oligonucleotides, each of said set of combination oligonucleotides comprising a sequence region corresponding to said plus strand of said first nucleotide sequence and further comprising a sequence region corresponding to said plus strand of said second nucleotide sequence;
(d) preparing a first oligonucleotide pool comprising oligonucleotides corresponding to said plus strand of said first nucleotide sequence and said plus strand of said second nucleotide sequence and said set of combination oligonucleotides;
(e) preparing a second oligonucleotide pool comprising said minus strands corresponding to said first and second nucleotide sequences; and
(f) assembling a collection of recombination products by triplet mixing of oligonucleotides of said first and said second oligonucleotide pools.

23. The method of claim 22, wherein each combination oligonucleotide comprises a 5′ portion corresponding to said first nucleotide sequence and a 3′ portion corresponding to said second nucleotide sequence.

24. The method of claim 22, wherein each combination oligonucleotides comprises a 3′ portion corresponding to said first nucleotide sequence and a 5′ portion corresponding to said second nucleotide sequence.


SEQUENCE LISTING (231 sequences)

SEQ ID NO: 1 - protein, 291 aa, Artificial Sequence
  synthetic construct
MSLNVKQSRIAIFSSCLISISFFSQANTKGIDEIKNLETDFNGRIGVYALDTGSGKSFSYRANERFPLCSSFKGFLAAAVLKGSQDNRLNLNQIVNYNTRSLEFHSPITTKYKDNGMSLGDMAAAALQYSDNGATNIILERYIGGPEGMTKFMRSIGDEDFRLDRWELDLNTAIPGDERDTSTPAAVAKSLKTLALGNILSEHEKETYQTWLKGNTTGAARIRASVPSDWVVGDKTGSCGAYGTANDYAVVWPKNRAPLIISVYTTKNEKEAKHEDKVIAEASRIAIDNLK

SEQ ID NO: 2 - protein, 284 aa, Artificial Sequence
  synthetic construct
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDVTRLDRWEPELNEAIPNDERDTTMPAAMATTLRKLLGELLTLASRQQLIDWMEADKVAGPLLRSALPAGWFIADKSGASKRGSRGIIAALGPDGKPSRIVVIYTTGSQATMDERNRQIAEIGASLIKHW

SEQ ID NO: 3 - protein, 118 aa, Artificial Sequence
  synthetic construct
EAIPNDERDTTMPAAMATTLRKLLTGELLTLASRQQLIDWMEADKVAGPLLRSALPAGWFIADKSGASKRGSRGIIAALGPDGKPSRIVVIYTTGSQATMDERNRQIAEIGASLIKHW

SEQ ID NO: 4 - DNA, 723 bp, Artificial Sequence
  synthetic construct
gaggttcacctgcagcagtctttggcagagcttgtgaggtcaggggcctcagtcaagttgtcctgcacagcttctggcttcaacattaaacactactatatgcactgggtgaaacagaggcctgaacagggcctggagtggattggatggattaatcctgagaatgttgatactgaatatgcccccaagttccagggcaaggccactatgactgcagacacatcctccaacacagcctacctgcagctcagcagcctgacatctgaggacactgccgtctattactgtaatcactataggtacgccgtagggggtgctttggactactggggtcaaggcaccacggtcaccgtctcctcaggtggaggcggttcaggcggaggtggctctggcggtggcggatcggacatcgagctcactcagtctccagcaatcatgtctgcatctccaggggagaaggtcaccatgacctgcagtgccagctcaagtgtaagttacatacactggtatcagcagaagtcaggcacctcccccaaaagatgggtttatgacacatccaaactggcttctggagtccctgctcgcttcagtggcagtgggtctgggacctcttactctctcacaatcagcaccatggaggctgaagtagctgccacttattactgccagcagtggaataataacccatacacgttcggaggagggaccaagctggaaataaaa

SEQ ID NO: 5 - protein, 241 aa, Artificial Sequence
  synthetic construct
EVHLQQSLAELVRSGASVKLSCTASGFNIKHYYMHWVKQRPEQGLEWIGWINPENVDTEYAPKFQGKATMTADTSSNTAYLQLSSLTSEDTAVYYCNHYRYAVGGALDYWGQGTTVTVSSGGGGSGGGGSGGGGSDIELTQSPAIMSASPGEKVTMTCSASSSVSYIHWYQQKSGTSPKRWVYDTSKLASGVPARFSGSGSGTSYSLTISTMEAEVAATYYCQQWNNNPYTFGGGTKLEIK

SEQ ID NO: 6 - DNA, 819 bp, Artificial Sequence
  synthetic construct
atggccgaggtgcagctggtggagtctgggggaggcctggtcaagcctggggggtccctgagactctcctgtgcagcctctggattcaccttcagtaactatagcatgaactgggtccgccaggctccagggaaggggctggagtgggtctcatccattagtagtagtagtagttacatatactacgcagacttcgtgaagggccgattcaccatctccagagacaacgccaagaactcactgtatctgcaaatgaacagcctgagagccgaggacacggctgtttattactgtgcgagatccagtattacgatttttggtggcggtatggacgtctggggcagaggcaccctggtcaccgtctcctcaggtggaggcggttcaggcggaggtggcagcggcggtggcggatcgcagtctgtgctgactcagcctgcctccgtgtctgggtctcctggacagtcgatcaccatctcctgcgctggaaccagcagtgacgttggtggttataactatgtctcctggtaccaacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctggccgtcctaggtgcggccgcagaacaaaaactcatctcagaagaggatctgaatggggccgcacatcaccatcatcaccattaa

SEQ ID NO: 7 - protein, 257 aa, Artificial Sequence
  synthetic construct
EVQLVESGGGLVKPGGSLRLSCAASGFTFSNYSMNWVRQAPGKGLEWVSSISSSSSYIYYADFVKGRFTISRDNAKNSLYLQMNSLRAEDTAVYYCARSSITIFGGGMDVWGRGTLVTVSSGGGGSGGGGSGGGGSQSVLTQPASVSGSPGQSITISCAGTSSDVGGYNYVSWYQQHPGKAPKLMIYEGSKRPSGVSNRFSGSKSGNTASLTISGLQAEDEADYYCSSYTTRSTRVFGGGTKLAVLGAAAEQKLISE

SEQ ID NO: 8 - protein, 240 aa, Artificial Sequence
  synthetic construct
EVHLQQSLAELVRSGASVKLSCTASGFNIKHYYMHWVKQRPEQGLEWIGWINPENVDTEYAPKFQGKATMTADTSSNTAYLQLSSLTSEDTAVYYCNHYRYAVGGALDYWGQGTTVTVSSGGGGSGGGGSGGGGSDIELTQSPAIMSASPGEKVTMTCSASSSVSYIHWYQQKSGTSPKRWVYDTSKLASGVPARFSGSGSGTSYSLTISTMEAEVAATYYCQQWNNNPYTFGGGTKLEI

SEQ ID NO: 9 - protein, 240 aa, Artificial Sequence
  synthetic construct
EVQLVESGGGLVKPGGSLRLSCAASGFTFSNYSMNWVRQAPGKGLEWVSSISSSSSYIYYADFVKGRFTISRDNAKNSLYLQMNSLRAEDTAVYYCARSSITIFGGGMDVWGRGTLVTVSSGGGGSGGGGSGGGGSQSVLTQPASVSGSPGQSITISCAGTSSDVGGYNYVSWYQQHPGKAPKLMIYEGSKRPSGVSNRFSGSKSGNTASLTISGLQAEDEADYYCSSYTTRSTRVFGGG

SEQ ID NO: 10 - DNA, 750 bp, Artificial Sequence
  synthetic construct
gaagtgcatctgcaacagagcctagcggaactggtacgttcaggcgcttcggtcaaactctcctgcaccgcaagtggatttaatattaaacactactatatgcattgggttaaacagaggccggagcaagggctggaatggatcggttggattaaccccgaaaatgtggacacagagtacgccccgaagttccagggcaaagcgactatgacggccgatacctctagcaacacggcatatcttcagctgtcgtcattgacttccgaagatacagctgtttattactgtaatcactatagatacgcggtcggtggcgcactggactattggggtcaagggaccacggtaaccgtgagttctggaggcggtggcagcggtggcgggggttccggcggaggcggttcggatatcgaattaactcagtcacctgccattatgagcgctagtccaggggagaaagttaccatgacatgctctgcgagctcctcggtcagttatatccattggtaccagcaaaaatcaggcacgtctccgaagcgatgggtgtatgataccagcaaactggcctctggtgttcctgcacggttttccggcagcggttcgggaactagttactcattaaccattagcacgatggaagcggaagtagccgctacctattactgtcagcagtggaacaataacccgtatacattcggcgggggtacgaaattggagatcgtagcgagtagcatttttttcatggtgtta

SEQ ID NO: 11 - DNA, 50 bp, Artificial Sequence
  synthetic construct
gaagtgcatctgcaacagagcctagcggaactggtacgttcaggcgcttc

SEQ ID NO: 12 - DNA, 50 bp, Artificial Sequence
  synthetic construct
ggtcaaactctcctgcaccgcaagtggatttaatattaaacactactata

SEQ ID NO: 13 - DNA, 50 bp, Artificial Sequence
  synthetic construct
tgcattgggttaaacagaggccggagcaagggctggaatggatcggttgg

SEQ ID NO: 14 - DNA, 50 bp, Artificial Sequence
  synthetic construct
attaaccccgaaaatgtggacacagagtacgccccgaagttccagggcaa

SEQ ID NO: 15 - DNA, 50 bp, Artificial Sequence
  synthetic construct
agcgactatgacggccgatacctctagcaacacggcatatcttcagctgt

SEQ ID NO: 16 - DNA, 50 bp, Artificial Sequence
  synthetic construct
cgtcattgacttccgaagatacagctgtttattactgtaatcactataga

SEQ ID NO: 17 - DNA, 50 bp, Artificial Sequence
  synthetic construct
tacgcggtcggtggcgcactggactattggggtcaagggaccacggtaac

SEQ ID NO: 18 - DNA, 50 bp, Artificial Sequence
  synthetic construct
cgtgagttctggaggcggtggcagcggtggcgggggttccggcggaggcg

SEQ ID NO: 19 - DNA, 50 bp, Artificial Sequence
  synthetic construct
gttcggatatcgaattaactcagtcacctgccattatgagcgctagtcca

SEQ ID NO: 20 - DNA, 50 bp, Artificial Sequence
  synthetic construct
ggggagaaagttaccatgacatgctctgcgagctcctcggtcagttatat

SEQ ID NO: 21 - DNA, 50 bp, Artificial Sequence
  synthetic construct
ccattggtaccagcaaaaatcaggcacgtctccgaagcgatgggtgtatg

SEQ ID NO: 22 - DNA, 50 bp, Artificial Sequence
  synthetic construct
ataccagcaaactggcctctggtgttcctgcacggttttccggcagcggt

SEQ ID NO: 23 - DNA, 50 bp, Artificial Sequence
  synthetic construct
tcgggaactagttactcattaaccattagcacgatggaagcggaagtagc

SEQ ID NO: 24 - DNA, 50 bp, Artificial Sequence
  synthetic construct
cgctacctattactgtcagcagtggaacaataacccgtatacattcggcg

SEQ ID NO: 25 - DNA, 50 bp, Artificial Sequence
  synthetic construct
ggggtacgaaattggagatcgtagcgagtagcatttttttcatggtgtta

SEQ ID NO: 26 - DNA, 25 bp, Artificial Sequence
  synthetic construct
ctaggctctgttgcagatgcacttc

SEQ ID NO: 27 - DNA, 50 bp, Artificial Sequence
  synthetic construct
acttgcggtgcaggagagtttgaccgaagcgcctgaacgtaccagttccg

SEQ ID NO: 28 - DNA, 50 bp, Artificial Sequence
  synthetic construct
tccggcctctgtttaacccaatgcatatagtagtgtttaatattaaatcc

SEQ ID NO: 29 - DNA, 50 bp, Artificial Sequence
  synthetic construct
ctgtgtccacattttcggggttaatccaaccgatccattccagcccttgc

SEQ ID NO: 30 - DNA, 50 bp, Artificial Sequence
  synthetic construct
agaggtatcggccgtcatagtcgctttgccctggaacttcggggcgtact

SEQ ID NO: 31 - DNA, 50 bp, Artificial Sequence
  synthetic construct
gctgtatcttcggaagtcaatgacgacagctgaagatatgccgtgttgct

SEQ ID NO: 32 - DNA, 50 bp, Artificial Sequence
  synthetic construct
agtccagtgcgccaccgaccgcgtatctatagtgattacagtaataaaca

SEQ ID NO: 33 - DNA, 50 bp, Artificial Sequence
  synthetic construct
gctgccaccgcctccagaactcacggttaccgtggtcccttgaccccaat

SEQ ID NO: 34 - DNA, 50 bp, Artificial Sequence
  synthetic construct
gactgagttaattcgatatccgaaccgcctccgccggaacccccgccacc

SEQ ID NO: 35 - DNA, 50 bp, Artificial Sequence
  synthetic construct
agcatgtcatggtaactttctcccctggactagcgctcataatggcaggt

SEQ ID NO: 36 - DNA, 50 bp, Artificial Sequence
  synthetic construct
gcctgatttttgctggtaccaatggatataactgaccgaggagctcgcag

SEQ ID NO: 37 - DNA, 50 bp, Artificial Sequence
  synthetic construct
acaccagaggccagtttgctggtatcatacacccatcgcttcggagacgt

SEQ ID NO: 38 - DNA, 50 bp, Artificial Sequence
  synthetic construct
tggttaatgagtaactagttcccgaaccgctgccggaaaaccgtgcagga

SEQ ID NO: 39 - DNA, 50 bp, Artificial Sequence
  synthetic construct
ccactgctgacagtaataggtagcggctacttccgcttccatcgtgctaa

SEQ ID NO: 40 - DNA, 50 bp, Artificial Sequence
  synthetic construct
gctacgatctccaatttcgtacccccgccgaatgtatacgggttattgtt

SEQ ID NO: 41 - DNA, 25 bp, Artificial Sequence
  synthetic construct
taacaccatgaaaaaaatgctactc

SEQ ID NO: 42 - DNA, 750 bp, Artificial Sequence
  synthetic construct
gaagtgcaactggtagaaagcggcggagggctagtcaaaccgggtggctcactgcgtctctcgtgcgcggcttccggttttaccttcagtaattactctatgaactgggttaggcaggcacccggcaaaggtctggagtgggtgagctcgatttcatccagttctagctatatctactatgccgactttgttaaagggagattcacaatttcccgagataatgcgaagaactcgctttatctgcagatgagttcattgcgggccgaagatactgcagtctactattgtgctcgcagcagtatcacgatttttggaggcggtatggacgtatggggccgtggtaccctggtgacggtttctagcggcgggggtggctccggaggcggtgggtcgggcggtggcggtagtcaatcagtcttaactcagccggcgtctgtgagcggatctcctggccagtccatcacaattagctgcgcagggacctcgagtgatgttggtggctacaactatgtatcatggtatcaacagcatccaggtaaagccccgaaactgatgatctacgaaggcagcaaacgcccttctggtgtgtccaatcgtttttcgggaagtaagagcgggaacacggcttcattaaccatttctggcttgcaggcggaggatgaagccgactattactgtagctcctatactacccgcagtacacgtgttttcggtggcggtgtagcgagtagcatttttttcatggtgtta

SEQ ID NO: 43 - DNA, 50 bp, Artificial Sequence
  synthetic construct
gaagtgcaactggtagaaagcggcggagggctagtcaaaccgggtggctc

SEQ ID NO: 44 - DNA, 50 bp, Artificial Sequence
  synthetic construct
actgcgtctctcgtgcgcggcttccggttttaccttcagtaattactcta

SEQ ID NO: 45 - DNA, 50 bp, Artificial Sequence
  synthetic construct
tgaactgggttaggcaggcacccggcaaaggtctggagtgggtgagctcg

SEQ ID NO: 46 - DNA, 50 bp, Artificial Sequence
  synthetic construct
atttcatccagttctagctatatctactatgccgactttgttaaagggag

SEQ ID NO: 47 - DNA, 50 bp, Artificial Sequence
  synthetic construct
attcacaatttcccgagataatgcgaagaactcgctttatctgcagatga

SEQ ID NO: 48 - DNA, 50 bp, Artificial Sequence
  synthetic construct
gttcattgcgggccgaagatactgcagtctactattgtgctcgcagcagt

SEQ ID NO: 49 - DNA, 50 bp, Artificial Sequence
  synthetic construct
atcacgatttttggaggcggtatggacgtatggggccgtggtaccctggt

SEQ ID NO: 50 - DNA, 50 bp, Artificial Sequence
  synthetic construct
gacggtttctagcggcgggggtggctccggaggcggtgggtcgggcggtg

SEQ ID NO: 51 - DNA, 50 bp, Artificial Sequence
  synthetic construct
gcggtagtcaatcagtcttaactcagccggcgtctgtgagcggatctcct

SEQ ID NO: 52 - DNA, 50 bp, Artificial Sequence
  synthetic construct
ggccagtccatcacaattagctgcgcagggacctcgagtgatgttggtgg

SEQ ID NO: 53 - DNA, 50 bp, Artificial Sequence
  synthetic construct
ctacaactatgtatcatggtatcaacagcatccaggtaaagccccgaaac

SEQ ID NO: 54 - DNA, 50 bp, Artificial Sequence
  synthetic construct
tgatgatctacgaaggcagcaaacgcccttctggtgtgtccaatcgtttt

SEQ ID NO: 55 - DNA, 50 bp, Artificial Sequence
  synthetic construct
tcgggaagtaagagcgggaacacggcttcattaaccatttctggcttgca

SEQ ID NO: 56 - DNA, 50 bp, Artificial Sequence
  synthetic construct
ggcggaggatgaagccgactattactgtagctcctatactacccgcagta

SEQ ID NO: 57 - DNA, 50 bp, Artificial Sequence
  synthetic construct
cacgtgttttcggtggcggtgtagcgagtagcatttttttcatggtgtta

SEQ ID NO: 58 - DNA, 25 bp, Artificial Sequence
  synthetic construct
cgccgctttctaccagttgcacttc

SEQ ID NO: 59 - DNA, 50 bp, Artificial Sequence
  synthetic construct
ggaagccgcgcacgagagacgcagtgagccacccggtttgactagccctc

SEQ ID NO: 60 - DNA, 50 bp, Artificial Sequence
  synthetic construct
ccgggtgcctgcctaacccagttcatagagtaattactgaaggtaaaacc

SEQ ID NO: 61 - DNA, 50 bp, Artificial Sequence
  synthetic construct
agatatagctagaactggatgaaatcgagctcacccactccagacctttg

SEQ ID NO: 62 - DNA, 50 bp, Artificial Sequence
  synthetic construct
cgcattatctcgggaaattgtgaatctccctttaacaaagtcggcatagt

SEQ ID NO: 63 - DNA, 50 bp, Artificial Sequence
  synthetic construct
gcagtatcttcggcccgcaatgaactcatctgcagataaagcgagttctt

SEQ ID NO: 64 - DNA, 50 bp, Artificial Sequence
  synthetic construct
ccataccgcctccaaaaatcgtgatactgctgcgagcacaatagtagact

SEQ ID NO: 65 - DNA, 50 bp, Artificial Sequence
  synthetic construct
gccacccccgccgctagaaaccgtcaccagggtaccacggccccatacgt

SEQ ID NO: 66 - DNA, 50 bp, Artificial Sequence
  synthetic construct
tgagttaagactgattgactaccgccaccgcccgacccaccgcctccgga

SEQ ID NO: 67 - DNA, 50 bp, Artificial Sequence
  synthetic construct
cgcagctaattgtgatggactggccaggagatccgctcacagacgccggc

SEQ ID NO: 68 - DNA, 50 bp, Artificial Sequence
  synthetic construct
ttgataccatgatacatagttgtagccaccaacatcactcgaggtccctg

SEQ ID NO: 69 - DNA, 50 bp, Artificial Sequence
  synthetic construct
cgtttgctgccttcgtagatcatcagtttcggggctttacctggatgctg

SEQ ID NO: 70 - DNA, 50 bp, Artificial Sequence
  synthetic construct
ccgtgttcccgctcttacttcccgaaaaacgattggacacaccagaaggg

SEQ ID NO: 71 - DNA, 50 bp, Artificial Sequence
  synthetic construct
gtaatagtcggcttcatcctccgcctgcaagccagaaatggttaatgaag

SEQ ID NO: 72 - DNA, 50 bp, Artificial Sequence
  synthetic construct
gctacaccgccaccgaaaacacgtgtactgcgggtagtataggagctaca

SEQ ID NO: 73 - DNA, 25 bp, Artificial Sequence
  synthetic construct
taacaccatgaaaaaaatgctactc

SEQ ID NO: 74 - DNA, 50 bp, Artificial Sequence
  synthetic construct
gaagtgcatctgcaacagagcctaggagggctagtcaaaccgggtggctc

SEQ ID NO: 75 - DNA, 50 bp, Artificial Sequence
  synthetic construct
ggtcaaactctcctgcaccgcaagtggttttaccttcagtaattactcta

SEQ ID NO: 76 - DNA, 50 bp, Artificial Sequence
  synthetic construct
tgcattgggttaaacagaggccggacaaaggtctggagtgggtgagctcg

SEQ ID NO: 77 - DNA, 50 bp, Artificial Sequence
  synthetic construct
attaaccccgaaaatgtggacacagactatgccgactttgttaaagggag

SEQ ID NO: 78 - DNA, 50 bp, Artificial Sequence
  synthetic construct
agcgactatgacggccgatacctctaagaactcgctttatctgcagatga

SEQ ID NO: 79 - DNA, 50 bp, Artificial Sequence
  synthetic construct
cgtcattgacttccgaagatacagcagtctactattgtgctcgcagcagt

SEQ ID NO: 80 - DNA, 50 bp, Artificial Sequence
  synthetic construct
tacgcggtcggtggcgcactggactacgtatggggccgtggtaccctggt

SEQ ID NO: 81 - DNA, 50 bp, Artificial Sequence
  synthetic construct
cgtgagttctggaggcggtggcagctccggaggcggtgggtcgggcggtg

SEQ ID NO: 82 - DNA, 50 bp, Artificial Sequence
  synthetic construct
gttcggatatcgaattaactcagtcgccggcgtctgtgagcggatctcct

SEQ ID NO: 83 - DNA, 50 bp, Artificial Sequence
  synthetic construct
ggggagaaagttaccatgacatgctcagggacctcgagtgatgttggtgg

SEQ ID NO: 84 - DNA, 50 bp, Artificial Sequence
  synthetic construct
ccattggtaccagcaaaaatcaggccagcatccaggtaaagccccgaaac

SEQ ID NO: 85 - DNA, 50 bp, Artificial Sequence
  synthetic construct
ataccagcaaactggcctctggtgtcccttctggtgtgtccaatcgtttt

SEQ ID NO: 86 - DNA, 50 bp, Artificial Sequence
  synthetic construct
tcgggaactagttactcattaaccacttcattaaccatttctggcttgca

SEQ ID NO: 87 - DNA, 50 bp, Artificial Sequence
  synthetic construct
cgctacctattactgtcagcagtggtgtagctcctatactacccgcagta

SEQ ID NO: 88 - DNA, 50 bp, Artificial Sequence; identical to SEQ ID NO: 25
  synthetic construct

SEQ ID NO: 89 - DNA, 50 bp, Artificial Sequence
  synthetic construct
gaagtgcaactggtagaaagcggcgcggaactggtacgttcaggcgcttc

SEQ ID NO: 90 - DNA, 50 bp, Artificial Sequence
  synthetic construct
actgcgtctctcgtgcgcggcttccggatttaatattaaacactactata

SEQ ID NO: 91 - DNA, 50 bp, Artificial Sequence
  synthetic construct
tgaactgggttaggcaggcacccgggcaagggctggaatggatcggttgg

SEQ ID NO: 92 - DNA, 50 bp, Artificial Sequence
  synthetic construct
atttcatccagttctagctatatctagtacgccccgaagttccagggcaa

SEQ ID NO: 93 - DNA, 50 bp, Artificial Sequence
  synthetic construct
attcacaatttcccgagataatgcgagcaacacggcatatcttcagctgt

SEQ ID NO: 94 - DNA, 50 bp, Artificial Sequence
  synthetic construct
gttcattgcgggccgaagatactgctgtttattactgtaatcactataga

SEQ ID NO: 95 - DNA, 50 bp, Artificial Sequence
  synthetic construct
atcacgatttttggaggcggtatggattggggtcaagggaccacggtaac

SEQ ID NO: 96 - DNA, 50 bp, Artificial Sequence
  synthetic construct
gacggtttctagcggcgggggtggcggtggcgggggttccggcggaggcg

SEQ ID NO: 97 - DNA, 50 bp, Artificial Sequence
  synthetic construct
gcggtagtcaatcagtcttaactcaacctgccattatgagcgctagtcca

SEQ ID NO: 98 - DNA, 50 bp, Artificial Sequence
  synthetic construct
ggccagtccatcacaattagctgcgctgcgagctcctcggtcagttatat

SEQ ID NO: 99 - DNA, 50 bp, Artificial Sequence
  synthetic construct
ctacaactatgtatcatggtatcaaacgtctccgaagcgatgggtgtatg

SEQ ID NO: 100 - DNA, 50 bp, Artificial Sequence
  synthetic construct
tgatgatctacgaaggcagcaaacgtcctgcacggttttccggcagcggt

SEQ ID NO: 101 - DNA, 50 bp, Artificial Sequence
  synthetic construct
tcgggaagtaagagcgggaacacggttagcacgatggaagcggaagtagc

SEQ ID NO: 102 - DNA, 50 bp, Artificial Sequence
  synthetic construct
ggcggaggatgaagccgactattacaacaataacccgtatacattcggcg

SEQ ID NO: 103 - DNA, 50 bp, Artificial Sequence; identical to SEQ ID NO: 57
  synthetic construct

SEQ ID NO: 104 - protein, 189 aa, Artificial Sequence
  synthetic construct
MQYLIVLALVAAASANVYHDGACPEVKPVDNFDWSNYHGKWWEVAKYPNSVEKYGKCGWAEYTPEGKSVKVSNYHVIHGKEYFIEGTAYPVGDSKIGKIYHKLTYGGVTKENVFNVLSTDNKNYIIGYYCKYDEDKKGHQDFVWVLSRSKVLTGEAKTAVENYLIGSPVVDSQKLVYSDFSEAACKVNN

SEQ ID NO: 105 - protein, 185 aa, Artificial Sequence
  synthetic construct
MESIMLFTLLGLCVGLAAGTEAAVVKDFDVNKFLGFWYEIALASKMGAYGLAHKEEKMGAMVVELKENLLALTTTYYNEGHCVLEKVAATQVDGSAKYKVTRISGEKEVVVVATDYMTYTVIDITSLVAGAVHRAMKLYSRSLDNNGEALNNFQKIALKHGFSETDIHILKHDLTCVNALQSGQI

SEQ ID NO: 106 - DNA, 24 bp, Artificial Sequence
  synthetic construct
tttttttttttttttttttttttt

SEQ ID NO: 107 - DNA, 48 bp, Artificial Sequence
  synthetic construct
tttttttttttttttttttttttttttttttttttttttttttttttt

SEQ ID NO: 108 - DNA, 50 bp, Artificial Sequence
  synthetic construct
atgcagctggcacgacaggtatgcagctggcacgacaggtatgcagctga

SEQ ID NO: 109 - DNA, 50 bp, Artificial Sequence
  synthetic construct
atgcagctggcacgacaggtatgcagctggcacgacaggtatgcagctgg

SEQ ID NO: 110 - DNA, 50 bp, Artificial Sequence
  synthetic construct
atgcagctggcacgacaggtatgcagctggcacgacaggtatgcagctgt

SEQ ID NO: 111 - DNA, 50 bp, Artificial Sequence
  synthetic construct
atgcagctggcacgacaggtatgcagctggcacgacaggtatgcagctgc

SEQ ID NO: 112 - DNA, 50 bp, Artificial Sequence
  synthetic construct
gaaagcggatgttgcgggttgttgttctgcgggttctgttcttcgttgac

SEQ ID NO: 113 - DNA, 50 bp, Artificial Sequence
  synthetic construct
atgaggttgccccgtattcaggaattctgtttggaaactgtcatgcagta

SEQ ID NO: 114 - DNA, 50 bp, Artificial Sequence
  synthetic construct
cctgatcgttctggcgctggttgcggcggcgtctgcgaacgtttaccacg

SEQ ID NO: 115 - DNA, 50 bp, Artificial Sequence
  synthetic construct
acggtgcgtgcccggaagttaaaccggttgacaacttcgactggtctaac

SEQ ID NO: 116 - DNA, 50 bp, Artificial Sequence
  synthetic construct
taccacggtaaatggtgggaagttgcgaaatacccgaactctgttgaaaa

SEQ ID NO: 117 - DNA, 50 bp, Artificial Sequence
  synthetic construct
atacggtaaatgcggttgggcggaatacaccccggaaggtaaatctgtta

SEQ ID NO: 118 - DNA, 50 bp, Artificial Sequence
  synthetic construct
aagtttctaactaccacgttatccacggtaaagaatacttcatcgaaggt

SEQ ID NO: 119 - DNA, 50 bp, Artificial Sequence
  synthetic construct
accgcgtacccggttggtgactctaaaatcggtaaaatctaccacaaact

SEQ ID NO: 120 - DNA, 50 bp, Artificial Sequence
  synthetic construct
gacctacggtggtgttaccaaagaaaacgttttcaacgttctgtctaccg

SEQ ID NO: 121 - DNA, 50 bp, Artificial Sequence
  synthetic construct
acaacaaaaactacatcatcggttactactgcaaatacgacgaagacaaa

SEQ ID NO: 122 - DNA, 50 bp, Artificial Sequence
  synthetic construct
aaaggtcaccaggacttcgtttgggttctgtctcgttctaaagttctgac

SEQ ID NO: 123 - DNA, 50 bp, Artificial Sequence
  synthetic construct
cggtgaagcgaaaaccgcggttgaaaactacctgatcggttctccggttg

SEQ ID NO: 124 - DNA, 50 bp, Artificial Sequence
  synthetic construct
ttgactctcagaaactggtttactctgacttctctgaagcggcgtgcaaa

SEQ ID NO: 125 - DNA, 50 bp, Artificial Sequence
  synthetic construct
gttaacaacactctcataccatggaagcttgcagtagcgagtagcatttt

SEQ ID NO: 126 - DNA, 50 bp, Artificial Sequence
  synthetic construct
tttcatggtgttattcccgatgctttttgaagttcgcagaatcgtatgtg

SEQ ID NO: 127 - DNA, 25 bp, Artificial Sequence
  synthetic construct
acaacaacccgcaacatccgctttc

SEQ ID NO: 128 - DNA, 50 bp, Artificial Sequence
  synthetic construct
attcctgaatacggggcaacctcatgtcaacgaagaacagaacccgcaga

SEQ ID NO: 129 - DNA, 50 bp, Artificial Sequence
  synthetic construct
cgcaaccagcgccagaacgatcaggtactgcatgacagtttccaaacaga

SEQ ID NO: 130 - DNA, 50 bp, Artificial Sequence
  synthetic construct
ggtttaacttccgggcacgcaccgtcgtggtaaacgttcgcagacgccgc

SEQ ID NO: 131 - DNA, 50 bp, Artificial Sequence
  synthetic construct
caacttcccaccatttaccgtggtagttagaccagtcgaagttgtcaacc

SEQ ID NO: 132 - DNA, 50 bp, Artificial Sequence
  synthetic construct
ttccgcccaaccgcatttaccgtatttttcaacagagttcgggtatttcg

SEQ ID NO: 133 - DNA, 50 bp, Artificial Sequence
  synthetic construct
tggataacgtggtagttagaaactttaacagatttaccttccggggtgta

SEQ ID NO: 134 - DNA, 50 bp, Artificial Sequence
  synthetic construct
tagagtcaccaaccgggtacgcggtaccttcgatgaagtattctttaccg

SEQ ID NO: 135 - DNA, 50 bp, Artificial Sequence
  synthetic construct
ttctttggtaacaccaccgtaggtcagtttgtggtagattttaccgattt

SEQ ID NO: 136 - DNA, 50 bp, Artificial Sequence
  synthetic construct
taaccgatgatgtagtttttgttgtcggtagacagaacgttgaaaacgtt

SEQ ID NO: 137 - DNA, 50 bp, Artificial Sequence
  synthetic construct
cccaaacgaagtcctggtgaccttttttgtcttcgtcgtatttgcagtag

SEQ ID NO: 138 - DNA, 50 bp, Artificial Sequence
  synthetic construct
ttcaaccgcggttttcgcttcaccggtcagaactttagaacgagacagaa

SEQ ID NO: 139 - DNA, 50 bp, Artificial Sequence
  synthetic construct
gagtaaaccagtttctgagagtcaacaaccggagaaccgatcaggtagtt

SEQ ID NO: 140 - DNA, 50 bp, Artificial Sequence
  synthetic construct
tccatggtatgagagtgttgttaactttgcacgccgcttcagagaagtca

SEQ ID NO: 141 - DNA, 50 bp, Artificial Sequence
  synthetic construct
aagcatcgggaataacaccatgaaaaaaatgctactcgctactgcaagct

SEQ ID NO: 142 - DNA, 25 bp, Artificial Sequence
  synthetic construct
cacatacgattctgcgaacttcaaa

SEQ ID NO: 143 - DNA, 50 bp, Artificial Sequence
  synthetic construct
ggttaggaaagcggatgttgcgggttgttgttctgcgggttctgttcttc

SEQ ID NO: 144 - DNA, 50 bp, Artificial Sequence
  synthetic construct
gttgacatgaggttgccccgtattcaggaattctgtttggaaactgtcat

SEQ ID NO: 145 - DNA, 50 bp, Artificial Sequence
  synthetic construct
ggaatctatcatgctgttcaccctgctgggtctgtgcgttggtctggcgg

SEQ ID NO: 146 - DNA, 50 bp, Artificial Sequence
  synthetic construct
cgggtaccgaagcggcggttgttaaagacttcgacgttaacaaattcctg

SEQ ID NO: 147 - DNA, 50 bp, Artificial Sequence
  synthetic construct
ggtttctggtacgaaatcgcgctggcgtctaaaatgggtgcgtacggtct

SEQ ID NO: 148 - DNA, 50 bp, Artificial Sequence
  synthetic construct
ggcgcacaaagaagaaaaaatgggtgcgatggttgttgaactgaaagaaa

SEQ ID NO: 149 - DNA, 50 bp, Artificial Sequence
  synthetic construct
acctgctggcgctgaccaccacctactacaacgaaggtcactgcgttctg

SEQ ID NO: 150 - DNA, 50 bp, Artificial Sequence
  synthetic construct
gaaaaagttgcggcgacccaggttgacggttctgcgaaatacaaagttac

SEQ ID NO: 151 - DNA, 50 bp, Artificial Sequence
  synthetic construct
ccgtatctctggtgaaaaagaagttgttgttgttgcgaccgactacatga

SEQ ID NO: 152 - DNA, 50 bp, Artificial Sequence
  synthetic construct
cctacaccgttatcgacatcacctctctggttgcgggtgcggttcaccgt

SEQ ID NO: 153 - DNA, 50 bp, Artificial Sequence
  synthetic construct
gcgatgaaactgtactctcgttctctggacaacaacggtgaagcgctgaa

SEQ ID NO: 154 - DNA, 50 bp, Artificial Sequence
  synthetic construct
caacttccagaaaatcgcgctgaaacacggtttctctgaaaccgacatcc

SEQ ID NO: 155 - DNA, 50 bp, Artificial Sequence
  synthetic construct
acatcctgaaacacgacctgacctgcgttaacgcgctgcagtctggtcag

SEQ ID NO: 156 - DNA, 50 bp, Artificial Sequence
  synthetic construct
atcactctcataccatggaagcttgcagtagcgagtagcatttttttcat

SEQ ID NO: 157 - DNA, 50 bp, Artificial Sequence
  synthetic construct
ggtgttattcccgatgctttttgaagttcgcagaatcgtatgtgtagaaa

SEQ ID NO: 158 - DNA, 25 bp, Artificial Sequence
  synthetic construct
acccgcaacatccgctttcctaacc

SEQ ID NO: 159 - DNA, 50 bp, Artificial Sequence
  synthetic construct
gaatacggggcaacctcatgtcaacgaagaacagaacccgcagaacaaca

SEQ ID NO: 160 - DNA, 50 bp, Artificial Sequence
  synthetic construct
cagggtgaacagcatgatagattccatgacagtttccaaacagaattcct

SEQ ID NO: 161 - DNA, 50 bp, Artificial Sequence
  synthetic construct
ttaacaaccgccgcttcggtacccgccgccagaccaacgcacagacccag

SEQ ID NO: 162 - DNA, 50 bp, Artificial Sequence
  synthetic construct
ccagcgcgatttcgtaccagaaacccaggaatttgttaacgtcgaagtct

SEQ ID NO: 163 - DNA, 50 bp, Artificial Sequence
  synthetic construct
acccattttttcttctttgtgcgccagaccgtacgcacccattttagacg

SEQ ID NO: 164 - DNA, 50 bp, Artificial Sequence
  synthetic construct
taggtggtggtcagcgccagcaggttttctttcagttcaacaaccatcgc

SEQ ID NO: 165 - DNA, 50 bp, Artificial Sequence
  synthetic construct
caacctgggtcgccgcaactttttccagaacgcagtgaccttcgttgtag

SEQ ID NO: 166 - DNA, 50 bp, Artificial Sequence
  synthetic construct
aacttctttttcaccagagatacgggtaactttgtatttcgcagaaccgt

SEQ ID NO: 167 - DNA, 50 bp, Artificial Sequence
  synthetic construct
gaggtgatgtcgataacggtgtaggtcatgtagtcggtcgcaacaacaac

SEQ ID NO: 168 - DNA, 50 bp, Artificial Sequence
  synthetic construct
gagaacgagagtacagtttcatcgcacggtgaaccgcacccgcaaccaga

SEQ ID NO: 169 - DNA, 50 bp, Artificial Sequence
  synthetic construct
tttcagcgcgattttctggaagttgttcagcgcttcaccgttgttgtcca

SEQ ID NO: 170 - DNA, 50 bp, Artificial Sequence
  synthetic construct
caggtcaggtcgtgtttcaggatgtggatgtcggtttcagagaaaccgtg

SEQ ID NO: 171 - DNA, 50 bp, Artificial Sequence
  synthetic construct
caagcttccatggtatgagagtgatctgaccagactgcagcgcgttaacg

SEQ ID NO: 172 - DNA, 50 bp, Artificial Sequence
  synthetic construct
ttcaaaaagcatcgggaataacaccatgaaaaaaatgctactcgctactg

SEQ ID NO: 173 - DNA, 25 bp, Artificial Sequence
  synthetic construct
tttctacacatacgattctgcgaac

SEQ ID NO: 174 - DNA, 50 bp, Artificial Sequence
  synthetic construct
gaaagcggatgttgcgggttgttgttgttgttctgcgggttctgttcttc

SEQ ID NO: 175 - DNA, 50 bp, Artificial Sequence
  synthetic construct
atgaggttgccccgtattcaggaataggaattctgtttggaaactgtcat

SEQ ID NO: 176 - DNA, 50 bp, Artificial Sequence
  synthetic construct
cctgatcgttctggcgctggttgcgctgggtctgtgcgttggtctggcgg

SEQ ID NO: 177 - DNA, 50 bp, Artificial Sequence
  synthetic construct
acggtgcgtgcccggaagttaaaccagacttcgacgttaacaaattcctg

SEQ ID NO: 178 - DNA, 50 bp, Artificial Sequence
  synthetic construct
taccacggtaaatggtgggaagttgcgtctaaaatgggtgcgtacggtct

SEQ ID NO: 179 - DNA, 50 bp, Artificial Sequence
  synthetic construct
atacggtaaatgcggttgggcggaagcgatggttgttgaactgaaagaaa

SEQ ID NO: 180 - DNA, 50 bp, Artificial Sequence
  synthetic construct
aagtttctaactaccacgttatccactacaacgaaggtcactgcgttctg

SEQ ID NO: 181 - DNA, 50 bp, Artificial Sequence
  synthetic construct
accgcgtacccggttggtgactctaacggttctgcgaaatacaaagttac

SEQ ID NO: 182 - DNA, 50 bp, Artificial Sequence
  synthetic construct
gacctacggtggtgttaccaaagaagttgttgttgcgaccgactacatga

SEQ ID NO: 183 - DNA, 50 bp, Artificial Sequence
  synthetic construct
acaacaaaaactacatcatcggttatctggttgcgggtgcggttcaccgt

SEQ ID NO: 184 - DNA, 50 bp, Artificial Sequence
  synthetic construct
aaaggtcaccaggacttcgtttgggtggacaacaacggtgaagcgctgaa

SEQ ID NO: 185 - DNA, 50 bp, Artificial Sequence
  synthetic construct
cggtgaagcgaaaaccgcggttgaacacggtttctctgaaaccgacatcc

SEQ ID NO: 186 - DNA, 50 bp, Artificial Sequence
  synthetic construct
ttgactctcagaaactggtttactccgttaacgcgctgcagtctggtcag

SEQ ID NO: 187 - DNA, 50 bp, Artificial Sequence
  synthetic construct
gttaacaacactctcataccatggacagtagcgagtagcatttttttcat

SEQ ID NO: 188 - DNA, 50 bp, Artificial Sequence
  synthetic construct
tttcatggtgttattcccgatgcttgttcgcagaatcgtatgtgtagaaa

SEQ ID NO: 189 - DNA, 50 bp, Artificial Sequence
  synthetic construct
attcctgaatacggggcaacctcatgaagaacagaacccgcagaacaaca

SEQ ID NO: 190 - DNA, 50 bp, Artificial Sequence
  synthetic construct
cgcaaccagcgccagaacgatcaggatgacagtttccaaacagaattcct

SEQ ID NO: 191 - DNA, 50 bp, Artificial Sequence
  synthetic construct
ggtttaacttccgggcacgcaccgtccgccagaccaacgcacagacccag

SEQ ID NO: 192 - DNA, 50 bp, Artificial Sequence
  synthetic construct
caacttcccaccatttaccgtggtacaggaatttgttaacgtcgaagtct

SEQ ID NO: 193 - DNA, 50 bp, Artificial Sequence
  synthetic construct
ttccgcccaaccgcatttaccgtatagaccgtacgcacccattttagacg

SEQ ID NO: 194 - DNA, 50 bp, Artificial Sequence
  synthetic construct
tggataacgtggtagttagaaactttttctttcagttcaacaaccatcgc

SEQ ID NO: 195 - DNA, 50 bp, Artificial Sequence
  synthetic construct
tagagtcaccaaccgggtacgcggtcagaacgcagtgaccttcgttgtag

SEQ ID NO: 196 - DNA, 50 bp, Artificial Sequence
  synthetic construct
ttctttggtaacaccaccgtaggtcgtaactttgtatttcgcagaaccgt

SEQ ID NO: 197 - DNA, 50 bp, Artificial Sequence
  synthetic construct
taaccgatgatgtagtttttgttgttcatgtagtcggtcgcaacaacaac

SEQ ID NO: 198 - DNA, 50 bp, Artificial Sequence
  synthetic construct
cccaaacgaagtcctggtgacctttacggtgaaccgcacccgcaaccaga

SEQ ID NO: 199 - DNA, 50 bp, Artificial Sequence
  synthetic construct
ttcaaccgcggttttcgcttcaccgttcagcgcttcaccgttgttgtcca

SEQ ID NO: 200 - DNA, 50 bp, Artificial Sequence
  synthetic construct
gagtaaaccagtttctgagagtcaaggatgtcggtttcagagaaaccgtg

SEQ ID NO: 201 - DNA, 50 bp, Artificial Sequence
  synthetic construct
tccatggtatgagagtgttgttaacctgaccagactgcagcgcgttaacg

SEQ ID NO: 202 - DNA, 50 bp, Artificial Sequence
  synthetic construct
aagcatcgggaataacaccatgaaaatgaaaaaaatgctactcgctactg

SEQ ID NO: 203 - DNA, 50 bp, Artificial Sequence
  synthetic construct
ggttaggaaagcggatgttgcgggttctgcgggttctgttcttcgttgac

SEQ ID NO: 204 - DNA, 50 bp, Artificial Sequence
  synthetic construct
gttgacatgaggttgccccgtattctctgtttggaaactgtcatgcagta

SEQ ID NO: 205 - DNA, 50 bp, Artificial Sequence
  synthetic construct
ggaatctatcatgctgttcaccctggcggcgtctgcgaacgtttaccacg

SEQ ID NO: 206 - DNA, 50 bp, Artificial Sequence
  synthetic construct
cgggtaccgaagcggcggttgttaaggttgacaacttcgactggtctaac

SEQ ID NO: 207 - DNA, 50 bp, Artificial Sequence
  synthetic construct
ggtttctggtacgaaatcgcgctggcgaaatacccgaactctgttgaaaa

SEQ ID NO: 208 - DNA, 50 bp, Artificial Sequence
  synthetic construct
ggcgcacaaagaagaaaaaatgggttacaccccggaaggtaaatctgtta

SEQ ID NO: 209 - DNA, 50 bp, Artificial Sequence
  synthetic construct
acctgctggcgctgaccaccacctacggtaaagaatacttcatcgaaggt

SEQ ID NO: 210 - DNA, 50 bp, Artificial Sequence
  synthetic construct
gaaaaagttgcggcgacccaggttgaaatcggtaaaatctaccacaaact

SEQ ID NO: 211 - DNA, 50 bp, Artificial Sequence
  synthetic construct
ccgtatctctggtgaaaaagaagttaacgttttcaacgttctgtctaccg

SEQ ID NO: 212 - DNA, 50 bp, Artificial Sequence
  synthetic construct
cctacaccgttatcgacatcacctcctactgcaaatacgacgaagacaaa

SEQ ID NO: 213 - DNA, 50 bp, Artificial Sequence
  synthetic construct
gcgatgaaactgtactctcgttctcttctgtctcgttctaaagttctgac

SEQ ID NO: 214 - DNA, 50 bp, Artificial Sequence
  synthetic construct
caacttccagaaaatcgcgctgaaaaactacctgatcggttctccggttg

SEQ ID NO: 215 - DNA, 50 bp, Artificial Sequence
  synthetic construct
acatcctgaaacacgacctgacctgtgacttctctgaagcggcgtgcaaa

SEQ ID NO: 216 - DNA, 50 bp, Artificial Sequence
  synthetic construct
atcactctcataccatggaagcttgagcttgcagtagcgagtagcatttt

SEQ ID NO: 217 - DNA, 50 bp, Artificial Sequence
  synthetic construct
ggtgttattcccgatgctttttgaatttgaagttcgcagaatcgtatgtg

SEQ ID NO: 218 - DNA, 50 bp, Artificial Sequence
  synthetic construct
gaatacggggcaacctcatgtcaacgtcaacgaagaacagaacccgcaga

SEQ ID NO: 219 - DNA, 50 bp, Artificial Sequence
  synthetic construct
cagggtgaacagcatgatagattcctactgcatgacagtttccaaacaga

SEQ ID NO: 220 - DNA, 50 bp, Artificial Sequence
  synthetic construct
ttaacaaccgccgcttcggtacccgcgtggtaaacgttcgcagacgccgc

SEQ ID NO: 221 - DNA, 50 bp, Artificial Sequence
  synthetic construct
ccagcgcgatttcgtaccagaaaccgttagaccagtcgaagttgtcaacc

SEQ ID NO: 222 - DNA, 50 bp, Artificial Sequence
  synthetic construct
acccattttttcttctttgtgcgccttttcaacagagttcgggtatttcg

SEQ ID NO: 223 - DNA, 50 bp, Artificial Sequence
  synthetic construct
taggtggtggtcagcgccagcaggttaacagatttaccttccggggtgta

SEQ ID NO: 224 - DNA, 50 bp, Artificial Sequence
  synthetic construct
caacctgggtcgccgcaactttttcaccttcgatgaagtattctttaccg

SEQ ID NO: 225 - DNA, 50 bp, Artificial Sequence
  synthetic construct
aacttctttttcaccagagatacggagtttgtggtagattttaccgattt

SEQ ID NO: 226 - DNA, 50 bp, Artificial Sequence
  synthetic construct
gaggtgatgtcgataacggtgtaggcggtagacagaacgttgaaaacgtt

SEQ ID NO: 227 - DNA, 50 bp, Artificial Sequence
  synthetic construct
gagaacgagagtacagtttcatcgctttgtcttcgtcgtatttgcagtag

SEQ ID NO: 228 - DNA, 50 bp, Artificial Sequence
  synthetic construct
tttcagcgcgattttctggaagttggtcagaactttagaacgagacagaa

SEQ ID NO: 229 - DNA, 50 bp, Artificial Sequence
  synthetic construct
caggtcaggtcgtgtttcaggatgtcaaccggagaaccgatcaggtagtt

SEQ ID NO: 230 - DNA, 50 bp, Artificial Sequence
  synthetic construct
caagcttccatggtatgagagtgattttgcacgccgcttcagagaagtca

SEQ ID NO: 231 - DNA, 50 bp, Artificial Sequence
  synthetic construct
ttcaaaaagcatcgggaataacaccaaaatgctactcgctactgcaagct